(19) United States
(12) Patent Application Publication
(10) Pub. No.: US 2024/0408134 A1
(43) Pub. Date: Dec. 12, 2024

(54) CELL

(71) Applicant: AUTOLUS LIMITED, London (GB)

(72) Inventors: Saket Srivastava, London (GB); Shaun Cordoba, London (GB); Shimobi Onuoha, London (GB); Simon Thomas, London (GB); Martin Pulé, London (GB)

(21) Appl. No.: 17/923,626

(22) PCT Filed: Apr. 6, 2021
(86) PCT No.: PCT/GB2021/051099

§ 371 Date: Nov. 7, 2022

(30) Foreign Application Priority Data
May 7, 2020 (GB) 2006820.1

(51) Int. Cl. A61K 35/17; A61K 39/00; A61P 35/02; C07K 14/705; C07K 14/71; C07K 14/725; C07K 16/28; C12N 5/0783; C12N 15/86

(52) U.S. Cl. CPC A61K 35/17; A61K 39/4611; A61K 39/4631; A61K 39/4637; A61K 39/464412; A61K 39/464413; A61P 35/02; C07K 14/7051; C07K 14/70521; C07K 14/70578; C07K 14/71; C07K 16/2803; C12N 5/0636; C12N 15/86; A61K 2239/13; A61K 2239/21; A61K 2239/28; A61K 2239/48; C07K 2317/565; C07K 2319/02; C07K 2319/03; C12N 2510/00; C12N 2740/15043

(57) ABSTRACT

The present invention relates to a cell which co-expresses: (i) a first chimeric antigen receptor (CAR) at the cell surface, comprising an antigen-binding domain which binds to CD19; (ii) a second CAR at the cell surface, comprising an antigen-binding domain which binds to CD22; (iii) dominant negative SHP2 (dSHP2); and (iv) dominant negative TGFβ receptor II (dnTGFβRII).

### CROSS-REFERENCE TO RELATED APPLICATIONS

This is a U.S. National Phase of International Application No. PCT/GB2021/051099, filed May 6, 2021, which claims priority to Great Britain Application No. 2006820.1, filed May 7, 2020.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “56892_Sub_SeqListing.txt”, which was created on May 2, 2023 and is 90,858 bytes in size. The subject matter of the Sequence Listing is incorporated herein in its entirety by reference.

### FIELD OF THE INVENTION

The present invention relates to a cell which comprises more than one chimeric antigen receptor (CAR).

### BACKGROUND TO THE INVENTION

A number of immunotherapeutic agents have been described for use in cancer treatment, including therapeutic monoclonal antibodies (mAbs), immunoconjugated mAbs, radioconjugated mAbs and bi-specific T-cell engagers.

Typically these immunotherapeutic agents target a single antigen: for instance, Rituximab targets CD20; Myelotarg targets CD33; and Alemtuzumab targets CD52.

The human CD19 antigen is a 95 kDa transmembrane glycoprotein belonging to the immunoglobulin superfamily. CD19 is expressed very early in B-cell differentiation and is only lost at terminal B-cell differentiation into plasma cells. Consequently, CD19 is expressed on all B-cell malignancies apart from multiple myeloma. Since loss of the normal B-cell compartment is an acceptable toxicity, CD19 is an attractive CAR target and clinical studies targeting CD19 with CARs have seen promising results.

A particular problem in the field of oncology is provided by the Goldie-Coldman hypothesis: which describes that the sole targeting of a single antigen may result in tumour escape by modulation of said antigen due to the high mutation rate inherent in most cancers. This modulation of antigen expression may reduce the efficacy of known immunotherapeutics, including those which target CD19.

Thus a problem with immunotherapeutics targeted against CD19 is that a B-cell malignancy may mutate and become CD19-negative. This may result in relapse with CD19-negative cancers which are not responsive to CD19 targeted therapeutics. For example, in one paediatric study, Grupp et al. reported that half of all relapses following CD19-targeted chimeric antigen receptor therapy for B-acute Lymphoblastic leukaemia (B-ALL) were due to CD19-negative disease (56^{th} American Society of Hematology Annual Meeting and Exposition).

There is thus a need for immunotherapeutic agents which are capable of targeting more than one cell surface structure to reflect the complex pattern of marker expression that is associated with many cancers, including CD19-positive cancers.

### Chimeric Antigen Receptors (CARs)

Chimeric antigen receptors are proteins which graft the specificity of, for example, a monoclonal antibody (mAb) to the effector function of a T-cell. Their usual form is that of a type I transmembrane domain protein with an antigen recognizing amino terminus, a spacer, a transmembrane domain all connected to a compound endodomain which transmits T-cell survival and activation signals (see FIG. 1A).

The most common form of these molecules are fusions of single-chain variable fragments (scFv) derived from monoclonal antibodies which recognize a target antigen, fused via a spacer and a trans-membrane domain to a signaling endodomain. Such molecules result in activation of the T-cell in response to recognition by the scFv of its target. When T cells express such a CAR, they recognize and kill target cells that express the target antigen. Several CARs have been developed against tumour associated antigens, and adoptive transfer approaches using such CAR-expressing T cells are currently in clinical trial for the treatment of various cancers.

It has been observed that using a CAR approach for cancer treatment, tumour heterogeneity and immunoediting can cause escape from CAR treatment. For example, in the study described by Grupp et al (2013; New Eng. J. Med 368:1509-1518, paper No 380, ASH 2014) CAR-modified T cell approach was used for the treatment of acute B-lymphocytic leukemia. In that clinical trial it was found that 10 patients with a complete remission after one month did relapse and 5 of them relapsed with CD19-negative disease.

There is thus a need for alternative CAR treatment approaches which address the problems of cancer escape and tumour heterogeneity.

### Expression of Two CAR Binding Specificities

Bispecific CARs known as tandem CARs or TanCARs have been developed in an attempt to target multiple cancer specific markers simultaneously. In a TanCAR, the extracellular domain comprises two antigen binding specificities in tandem, joined by a linker. The two binding specificities (scFvs) are thus both linked to a single transmembrane portion: one scFv being juxtaposed to the membrane and the other being in a distal position.

Grada et al (2013, Mol Ther Nucleic Acids 2:e105) describes a TanCAR which includes a CD19-specific scFv, followed by a Gly-Ser linker and then a HER2-specific scFv. The HER2-scFv was in the juxta-membrane position, and the CD19-scFv in the distal position. The Tan CAR was shown to induce distinct T cell reactivity against each of the two tumour restricted antigens. This arrangement was chosen because the respective lengths of HER2 (632 aa/125 Å) and CD19 (280aa, 65 Å) lends itself to that particular spatial arrangement. It was also known that the HER2 scFv bound the distal-most 4 loops of HER2.

The problem with this approach is that the juxta-membrane scFv may be inaccessible due to the presence of the distal scFv, especially which it is bound to the antigen. In view of the need to choose the relative positions of the two scFvs in view of the spatial arrangement of the antigen on the target cell, it may not be possible to use this approach for all scFv binding pairs. Moreover, it is unlikely that the TanCar approach could be used for more than two scFvs, a TanCAR with three or more scFvs would be a very large molecule and the scFvs may well fold back on each other, obscuring the antigen-binding sites. It is also doubtful that antigen-binding by the most distal scFv, which is separated from the transmembrane domain by two or more further scFvs, would be capable of triggering T cell activation.

There is thus a need for an alternative approach to express two CAR binding specificities on the surface of a cell such as a T cell. This problem was addressed by the present inventors in WO2016/102965. There remains a need to provide cells expressing two CAR binding specificities on the surface that also exhibit improved survival and persistence.

### SUMMARY OF THE INVENTION

The present inventors have developed a CAR T cell which expresses two CARs at the cell surface, one specific for CD19 and one specific for CD22. Furthermore, the CAR T cells of the invention additionally comprise enhancement modules, which are described in more detail herein.

Thus in a first aspect the present invention provides a cell which co-expresses a first chimeric antigen receptor (CAR) and second CAR at the cell surface, each CAR comprising an antigen-binding domain, wherein the antigen-binding domain of the first CAR binds to CD19 and the antigen-binding domain of the second CAR binds to CD22, further wherein the cell expresses dominant negative SHP2 (dSHP2) and dominant negative TGFβ receptor II (dnTGFβRII).

The fact the one CAR binds CD19 and the other CAR binds CD22 is advantageous because some lymphomas and leukaemias become CD19 negative after CD19 targeting, (or possibly CD22 negative after CD22 targeting), so it gives a “back-up” antigen, should this occur. Additionally, the present inventors have shown that the particular combination with dSHP2 and dnTGFβRII is advantageous, as further described herein.

The present inventors have also shown that a particular combination of intracellular signalling domains is also advantageous. Accordingly, there is provided a cell of the invention in which each CAR comprises an intracellular signalling domain, wherein the intracellular signalling domain of the first CAR comprises a TNF receptor family endodomain; and the intracellular signalling domain of the second CAR comprises a co-stimulatory endodomain.

The co-stimulatory endodomain may be a CD28 co-stimulatory endodomain. Examples of suitable TNF receptor family endodomains include, but are not limited to, OX-40 and 4-1BB endodomain.

The intracellular signalling domain of the first and the second CAR may also comprise an ITAM-containing endodomain.

The cell may be an immune effector cell, such as a T-cell, natural killer (NK) cell, or NKT cell. Features mentioned herein in connection with a T cell apply equally to other immune effector cells, such as NK cells or NKT cells.

Each CAR may comprise:
(i) an antigen-binding domain;
(ii) a spacer; and
(iii) a trans-membrane domain.

Each CAR may comprise:
(i) an antigen-binding domain;
(ii) a spacer;
(iii) a trans-membrane domain; and
(iv) an endodomain.

The spacer of the first CAR may be different to the spacer of the second CAR, such the first and second CAR do not form heterodimers.

The spacer of the first CAR may have a different length and/or configuration from the spacer of the second CAR, such that each CAR is tailored for recognition of its respective target antigen. A suitable spacer for the second CAR includes, but is not limited to, cartilage oligomeric matrix protein (COMP) coiled coil domain.

The antigen-binding domain of the second CAR may bind to a membrane-distal epitope on CD22. The antigen-binding domain of the second CAR may bind to an epitope on Ig domain 7, 6, 5 or 4 of CD22, for example on Ig domain 5 of CD22.

The antigen-binding domain of the first CAR may bind to an epitope on CD19 which is encoded by exon 1, 3 or 4.

The CD19-binding domain of the first CAR may comprise:
a) a heavy chain variable region (VH) having complementarity determining regions (CDRs) with the following sequences:

CDR1-
(SEQ ID No. 1)
SYWMN;
CDR2-
(SEQ ID NO. 2)
QIWPGDGDTNYNGKFK
CDR3-
(SEQ ID No. 3)
RETTTVGRYYYAMDY;

and
b) a light chain variable region (VL) having CDRs with the following sequences:

CDR1-
(SEQ ID No. 4)
KASQSVDYDGDSYLN;
CDR2-
(SEQ ID No. 5)
DASNLVS
CDR3-
(SEQ ID NO. 6)
QQSTEDPWT.

The CD19 binding domain may comprise a VH domain having the sequence shown as SEQ ID No. 7, or SEQ ID NO 8; or a VL domain having the sequence shown as SEQ ID No 9, SEQ ID No. 10 or SEQ ID No. 11 or a variant thereof having at least 90% sequence identity which retains the capacity to bind CD19.

The CD19 binding domain may comprise the sequence shown as SEQ ID No 12, SEQ ID No. 13 or SEQ ID No. 14 or a variant thereof having at least 90% sequence identity which retains the capacity to bind CD19.

The CD22-binding domain of the second CAR may comprises:
a) a heavy chain variable region (VH) having complementarity determining regions (CDRs) with the following sequences:

CDR1-
(SEQ ID No. 15)
NYWIN;
CDR2-
(SEQ ID NO. 16)
NIYPSDSFTNYNQKFKD
CDR3-
(SEQ ID No. 17)
DTQERSWYFDV;

and
b) a light chain variable region (VL) having CDRs with the following sequences:

CDR1-
(SEQ ID No. 18)
RSSQSLVHSNGNTYLH;
CDR2-
(SEQ ID No. 19)
KVSNRFS
CDR3-
(SEQ ID NO. 20)
SQSTHVPWT.

The CD22 binding domain may comprise a VH domain having the sequence shown as SEQ ID No. 21, or SEQ ID NO 22; or a VL domain having the sequence shown as SEQ ID No 23, or SEQ ID No. 24 or a variant thereof having at least 90% sequence identity which retains the capacity to bind CD22.

The CD22 binding domain may comprise the sequence shown as SEQ ID No 25 or SEQ ID No. 26 or a variant thereof having at least 90% sequence identity which retains the capacity to bind CD22.

The endodomain of the second CAR may comprise a co-stimulatory domain and an ITAM-containing domain; and the endodomain of the first CAR may comprise a TNF receptor family domain and an ITAM-containing domain.

For example, the first CAR (which is CD19-specific) may have the structure:

AgB1-spacer1-TM1-TNF-ITAM
in which:
AgB1 is the antigen-binding domain;
spacer1 is the spacer;
TM1 is the transmembrane domain;
TNF is a TNF receptor endodomain; and
ITAM is an ITAM-containing endodomain;
and the second CAR (which is CD22-specific) may have the structure:

AgB2-spacer2-TM2-costim-ITAM
in which:
AgB2 is the antigen-binding domain;
spacer2 is the spacer;
TM2 is the transmembrane domain;
costim is a co-stimulatory domain; and
ITAM is an ITAM-containing endodomain.

In a second aspect, the present invention provides, a nucleic acid sequence encoding both the first and second chimeric antigen receptors (CARs) as defined in the first aspect of the invention, together with dSHP2, and dnTGFβRII.

The nucleic acid sequence may have the following structure:

module1-coexpr-AgB1-spacer1-TM1-coexpr-AgB2-spacer2-TM2-coexpr-module2
in which
AgB1 is a nucleic acid sequence encoding the antigen-binding domain of the first CAR; spacer1 is a nucleic acid sequence encoding the spacer of the first CAR;
TM1 is a a nucleic acid sequence encoding the transmembrane domain of the first CAR; coexpr is a nucleic acid sequence enabling co-expression
AgB2 is a nucleic acid sequence encoding the antigen-binding domain of the second CAR;
spacer2 is a nucleic acid sequence encoding the spacer of the second CAR;
TM2 is a a nucleic acid sequence encoding the transmembrane domain of the second CAR;
module1 and module2 are nucleic acid sequences encoding either dominant negative SHP2 (dSHP2) or dominant negative TGFβRII (dnTGFβRII), wherein when module1 encodes dSHP2 module2 encodes dnTGFβRII and when module2 encodes dnTGFβRII module1 encodes dSHP2;
which nucleic acid sequence, when expressed in a T cell, encodes a polypeptide which is cleaved at the cleavage site such that the first and second CARs are co-expressed at the T cell surface.

The nucleic acid sequence may have the following structure:

module1-coexpr-AgB1-spacer1-TM1-endo1-coexpr-AbB2-spacer2-TM2-endo2-coexpr-module2
in which
AgB1 is a nucleic acid sequence encoding the antigen-binding domain of the first CAR;
spacer1 is a nucleic acid sequence encoding the spacer of the first CAR;
TM1 is a a nucleic acid sequence encoding the transmembrane domain of the first CAR;
endo1 is a nucleic acid sequence encoding the endodomain of the first CAR;
coexpr is a nucleic acid sequence enabling co-expression
AgB2 is a nucleic acid sequence encoding the antigen-binding domain of the second CAR;
spacer2 is a nucleic acid sequence encoding the spacer of the second CAR;
TM2 is a a nucleic acid sequence encoding the transmembrane domain of the second CAR;
endo2 is a nucleic acid sequence encoding the endodomain of the second CAR;
module1 and module2 are nucleic acid sequences encoding either dominant negative SHP2 (dSHP2) or dominant negative TGFβRII (dnTGFβRII), wherein when module1 encodes dSHP2 module2 encodes dnTGFβRII and when module2 encodes dnTGFβRII module1 encodes dSHP2;
which nucleic acid sequence, when expressed in a T cell, encodes a polypeptide which is cleaved at the cleavage site such that the first and second CARs are co-expressed at the T cell surface.

The nucleic acid sequence allowing co-expression of two CARs may encode a self-cleaving peptide or a sequence which allows alternative means of co-expressing two CARs such as an internal ribosome entry sequence or a 2^{nd} promoter or other such means whereby one skilled in the art can express two proteins from the same vector.

Alternative codons may be used in regions of sequence encoding the same or similar amino acid sequences, such as the transmembrane and/or intracellular T cell signalling domain (endodomain) in order to avoid homologous recombination. For example, alternative codons may be used in the portions of sequence encoding the spacer, the transmembrane domain and/or all or part of the endodomain, such that the two CARs have the same or similar amino acid sequences for this or these part(s) but are encoded by different nucleic acid sequences.

In a third aspect, the present invention provides kit which comprises
(i) a first nucleic acid sequence encoding the first chimeric antigen receptor (CAR), which nucleic acid sequence has the following structure:

AgB1-spacer1-TM1
in which
AgB1 is a nucleic acid sequence encoding the antigen-binding domain of the first CAR which binds to CD19;
spacer1 is a nucleic acid sequence encoding the spacer of the first CAR;
TM1 is a nucleic acid sequence encoding the transmembrane domain of the first CAR;
(ii) a second nucleic acid sequence encoding the second chimeric antigen receptor, which nucleic acid sequence has the following structure:

AgB2-spacer2-TM2
in which
AgB2 is a nucleic acid sequence encoding the antigen-binding domain of the second CAR which binds to CD22;
spacer2 is a nucleic acid sequence encoding the spacer of the second CAR; and
TM2 is a nucleic acid sequence encoding the transmembrane domain of the second CAR; and
(iii) a third nucleic acid sequence encoding dSHP2 and dnTGFβRII as described herein.

The kit may comprise
(i) a first nucleic acid sequence encoding the first chimeric antigen receptor (CAR), which nucleic acid sequence has the following structure:

AgB1-spacer1-TM1-endo1
in which
AgB1 is a nucleic acid sequence encoding the antigen-binding domain of the first CAR;
spacer1 is a nucleic acid sequence encoding the spacer of the first CAR;
TM1 is a nucleic acid sequence encoding the transmembrane domain of the first CAR;
endo1 is a nucleic acid sequence encoding the endodomain of the first CAR; and
(ii) a second nucleic acid sequence encoding the second chimeric antigen receptor (CAR), which nucleic acid sequence has the following structure:

AgB2-spacer2-TM2-endo2
in which
AgB2 is a nucleic acid sequence encoding the antigen-binding domain of the second CAR;
spacer2 is a nucleic acid sequence encoding the spacer of the second CAR;
TM2 is a nucleic acid sequence encoding the transmembrane domain of the second CAR;
endo2 is a nucleic acid sequence encoding the endodomain of the second CAR.

In a fourth aspect, the present invention provides a kit comprising: a first vector which comprises the first nucleic acid sequence; a second vector which comprises the second nucleic acid sequence; and a third vector which comprises the third nucleic acid sequence.

The vectors may be plasmid vectors, retroviral vectors or transposon vectors. The vectors may be lentiviral vectors.

In a fifth aspect, the present invention provides a vector comprising a nucleic acid sequence according to the second aspect of the invention. The vector may be a lentiviral vector.

The vector may be a plasmid vector, a retroviral vector or a transposon vector.

In a sixth aspect the present invention provides a method for making a cell according to the first aspect of the invention, which comprises the step of introducing one or more nucleic acid sequence(s) encoding the first and second CARs, dSHP2, and dnTGFβRII; or one or more vector(s), as defined above, into a cell. The cell may be a T cell.

The cell may be from a sample isolated from a subject, including but not limited to a patient, a related or unrelated haematopoietic transplant donor, a completely unconnected donor, from cord blood, differentiated from an embryonic cell line, differentiated from an inducible progenitor cell line, or derived from a transformed cell line.

In a seventh aspect, the present invention provides a pharmaceutical composition comprising a plurality of cells according to the first aspect of the invention.

In an eighth aspect the present invention provides a method for treating and/or preventing a disease, which comprises the step of administering a pharmaceutical composition according to the seventh aspect of the invention to a subject.

The method may comprise the following steps:
(i) isolation of a cell-containing sample from a subject;
(ii) transduction or transfection of the cells with one or more nucleic acid sequence(s) encoding the first and second CAR, dSHP2, and dnTGFβRII, or one or more vector(s) comprising such nucleic acid sequence(s); and
(iii) administering the cells from (ii) to the subject.

The disease may be cancer. The cancer may be a B cell malignancy.

In a ninth aspect the present invention provides a pharmaceutical composition according to the seventh aspect of the invention for use in treating and/or preventing a disease.

In a tenth aspect the present invention provides the use of a cell according to the first aspect of the invention in the manufacture of a medicament for treating and/or preventing a disease.

The present invention also provides a nucleic acid sequence which comprises:
a) a first nucleotide sequence encoding a first chimeric antigen receptor (CAR);
b) a second nucleotide sequence encoding a second CAR;
wherein one CAR binds CD19 and the other CAR binds CD22; and
c) a sequence encoding a self-cleaving peptide positioned between the first and second nucleotide sequences, such that the two CARs are expressed as separate entities.

Alternative codons may be used in one or more portion(s) of the first and second nucleotide sequences in regions which encode the same or similar amino acid sequence(s).

The present invention also provides a vector and a cell comprising such a nucleic acid.

The vector may be a plasmid vector, a retroviral vector or a transposon vector.

The present inventors have also found that, in an OR gate system, performance is improved if the co-stimulatory domain and domain producing survival signals are “split” between the two (or more) CARs.

Thus, in a eleventh aspect there is provided a cell which co-expresses at the cell surface a first chimeric antigen receptor (CAR) comprising an antigen-binding domain which binds to CD19 and second CAR comprising an antigen-binding domain which binds to CD22, each CAR comprising an intracellular signalling domain, wherein the intracellular signalling domain of the first CAR comprises a TNF receptor family endodomain; and the intracellular signalling domain of the second CAR comprises a co-stimulatory domain.

The co-stimulatory domain may be a CD28 co-stimulatory domain. The TNF receptor family endodomain may be, for example, OX-40 or 4-1BB endodomain.

The intracellular signalling domain of the first and the second CAR may also comprise an ITAM-containing domain, such as a CD3 zeta endodomain.

The first CAR may have the structure:

AgB1-spacer1-TM1-TNF-ITAM
in which:
AgB1 is the antigen-binding domain of the first CAR;
spacer1 is the spacer of the first CAR;
TM1 is the transmembrane domain of the first CAR;
TNF is a TNF receptor endodomain; and
ITAM is an ITAM-containing endodomain.

The second CAR may have the structure:

AgB2-spacer2-TM2-costim-ITAM
in which:
AgB2 is the antigen-binding domain of the second CAR;
spacer2 is the spacer of the second CAR;
TM2 is the transmembrane domain of the second CAR;
costim is a co-stimulatory domain; and
ITAM is an ITAM-containing endodomain.

In a twelfth aspect there is provided a nucleic acid sequence encoding both the first and second chimeric antigen receptors (CARs) as defined in the eleventh aspect of the invention.

The nucleic acid sequence may have the following structure:

AgB1-spacer1-TM1-TNF-ITAM1-coexpr-AbB2-spacer2-TM2-costim-ITAM2
in which
AgB1 is a nucleic acid sequence encoding the antigen-binding domain of the first CAR;
spacer1 is a nucleic acid sequence encoding the spacer of the first CAR;
TM1 is a a nucleic acid sequence encoding the transmembrane domain of the first CAR;
TNF is a nucleic acid sequence encoding a TNF receptor endodomain;
ITAM1 is a nucleic acid sequence encoding the ITAM-containing endodomain of the first CAR;
coexpr is a nucleic acid sequence enabling co-expression
AgB2 is a nucleic acid sequence encoding the antigen-binding domain of the second CAR;
spacer2 is a nucleic acid sequence encoding the spacer of the second CAR;
TM2 is a nucleic acid sequence encoding the transmembrane domain of the second CAR;
costim is a nucleic acid sequence encoding a co-stimulatory domain;
ITAM2 is a nucleic acid sequence encoding the ITAM-containing endodomain of the second CAR.

When the nucleic acid sequence is expressed in a cell it may encode a polypeptide which is cleaved at the cleavage site such that the first and second CARs are co-expressed at the cell surface.

In a thirteenth aspect, there is provided a kit which comprises
(i) a first nucleic acid sequence encoding the first chimeric antigen receptor (CAR) as defined in the eleventh aspect of the invention, which nucleic acid sequence has the following structure:

AgB1-spacer1-TM1-TNF-ITAM1
in which
AgB1 is a nucleic acid sequence encoding the antigen-binding domain of the first CAR;
spacer1 is a nucleic acid sequence encoding the spacer of the first CAR;
TM1 is a nucleic acid sequence encoding the transmembrane domain of the first CAR;
TNF is a nucleic acid sequence encoding a TNF receptor endodomain;
ITAM1 is a nucleic acid sequence encoding the ITAM-containing endodomain of the first CAR; and
(ii) a second nucleic acid sequence encoding the second chimeric antigen receptor (CAR) as defined in the eleventh aspect of the invention, which nucleic acid sequence has the following structure:

AgB2-spacer2-TM2-costim-ITAM2
AgB2 is a nucleic acid sequence encoding the antigen-binding domain of the second CAR;
spacer2 is a nucleic acid sequence encoding the spacer of the second CAR;
TM2 is a nucleic acid sequence encoding the transmembrane domain of the second CAR;
costim is a nucleic acid sequence encoding a co-stimulatory domain; and
ITAM2 is a nucleic acid sequence encoding the ITAM-containing endodomain of the second CAR.

In a fourteenth aspect there is provided a vector comprising a nucleic acid sequence according to the eleventh aspect of the invention or as defined in the twelfth aspect of the invention.

In a fifteenth aspect, there is provided a method for making a cell according to the eleventh aspect of the invention, which comprises the step of introducing: a nucleic acid sequence according to twelfth aspect of the invention; a first nucleic acid sequence and a second nucleic acid sequence as defined in the thirteenth aspect of the invention; or a vector according to the fourteenth aspect of the invention, into a cell.

In a sixteenth aspect, the present invention provides a pharmaceutical composition comprising a plurality of cells according to the eleventh aspect of the invention.

There is also provided a method for treating and/or preventing a disease, which comprises the step of administering a pharmaceutical composition according to the sixteenth aspect of the invention to a subject.

There is also provided a pharmaceutical composition according to the sixteenth aspect of the invention for use in treating and/or preventing a disease.

There is also provided the use of a cell according to the eleventh aspect of the invention in the manufacture of a medicament for treating and/or preventing a disease.

By providing one CAR which targets CD19 and one CAR which targets CD22, it is possible to target each of these markers, thereby reducing the problem of cancer escape.

Because the CARs are expressed on the surface of the cell as separate molecules, this approach overcomes the spatial and accessibility issues associated with TanCARs. Cell activation efficiency is also improved. If each CAR has its own spacer, it is possible to tailor the spacer and therefore the distance that the binding domain projects from the cell surface and its flexibility etc. to the particular target antigen. This choice is unfettered by the design considerations associated with TanCARs, i.e. that one CAR needs to be juxtaposed to the T cell membrane and one CAR needs to be distal, positioned in tandem to the first CAR.

By providing a single nucleic acid which encodes the two CARs separated by a cleavage site, it is possible to engineer cells to co-express the two CARs using a simple single transduction procedure. A double transfection procedure could be used with CAR-encoding sequences in separate constructs, but this would be more complex and expensive and requires more integration sites for the nucleic acids. A double transfection procedure would also be associated with uncertainty as to whether both CAR-encoding nucleic acids had been transduced and expressed effectively.

The CARs will have portions of high homology, for example the transmembrane and/or intracellular signalling domains are likely to be highly homologous. If the same or similar linkers are used for the two CARs, then they will also be highly homologous. This would suggest that an approach where both CARs are provided on a single nucleic acid sequence would be inappropriate, because of the likelihood of homologous recombination between the sequences. However, the present inventors have found that by “codon wobbling” the portions of sequence encoding areas of high homology, it is possible to express two CARs from a single construct with high efficiency. Codon wobbling involves using alternative codons in regions of sequence encoding the same or similar amino acid sequences.

### DESCRIPTION OF THE FIGURES

FIG. 1: a) Schematic diagram illustrating a classical CAR. (b) to (d): Different generations and permutations of CAR endodomains: (b) initial designs transmitted ITAM signals alone through FcεR1-γ or CD3ζ endodomain, while later designs transmitted additional (c) one or (d) two co-stimulatory signals in the same compound endodomain.

FIG. 2: B-cell maturation pathway/B-cell ontogeny. DR=HLA-DR; cCD79=cytoplasmic CD79; cCD22=cytoplasmic CD22. Both CD19 and CD22 antigens are expressed during early stages in B-cell maturation. It is these cells that develop into B-cell acute leukaemias. Targeting both CD19 as well as CD22 simultaneously is most suited for targeting B-cell acute leukaemias.

FIG. 3: CD19 structure and exons

FIG. 4: Strategies for design of an anti-CD19 OR CD22 CAR cassette. Binders which recognize CD19 and binders which recognize CD22 are selected. An optimal spacer domain and signalling domain is selected for each CAR. (a) an OR gate cassette is constructed so that both CARs are co-expressed using a FMD-2A peptide. Any homologous sequences are codon-wobbled to avoid recombination. (b) The two CARs are co-expressed as separate proteins on the T-cell surface.

FIG. 5: Example of codon-wobbling to allow co-expression in a retroviral vector of identical peptide sequences but avoiding homologous recombination. Here, wild-type HCH2CH3-CD28tmZeta is aligned with codon-wobbled HCH2CH3-CD28tmZeta.

FIG. 6: Schematic of a CD19/CD22 OR GATE of the present invention.

FIG. 7: Naturally occurring dimeric, trimeric and tetrameric coiled coil structures (modified from Andrei N. Lupas and Markus Gruber; Adv Protein Chem. 2005; 70:37-78)

FIG. 8: Crystal structure of the pentameric coiled coil motif from collagen oligomeric matrix protein (COMP) and human IgG1. Individual chains are depicted with different colours. The coiled coil COMP structure is displayed from the N-terminus with the C-terminus extending into the page and also displayed from the profile with the C-terminus left to the N-terminus right. The human IgG1 is displayed from the profile with the N-terminus (top) to C-terminus (bottom).

FIG. 9: Truncation of the COMP spacer
a) schematic diagram showing the anti-ROR-1 COMP CAR, the COMP spacer was truncated from the N-terminus from 45 amino acids to “x” amino acids
b) 293T cells were transfected with the truncated constructs and analysed by FACS.

FIG. 10: Schematic diagram illustrating the mechanism of a) T-cell activation and b) T-cell inhibition in vivo

FIG. 11: Summary of CD19/CD22 OR gate constructs. The CD19 and CD22 CARs were separated by a self-cleaving 2A sequence in order to achieve expression of each CAR as a distinct molecule.

FIG. 12: Comparison of various CD19/CD22 OR gate constructs. Cells expressing the one of the constructs were co-cultured for 72 hours with target cells at a 1:1 effector:target (E:T) cell ratio (50,000 target cells). (A) Remaining target cells; (B) IL-2 production; (C) IFN-γ production; (D) Proliferation. Blue circles: Non-transduced cells; red squares: Construct 1; green diamonds: Construct 3; purple circles: Construct 4; black squares; Construct 5.

FIG. 13: In vitro testing of various CD19/CD22 OR gate constructs. Cells expressing the one of the constructs were co-cultured for 72 hours with target cells at a 1:1 and 1:10 effector:target (E:T) cell ratio. Blue circles: Non-transduced cells; red squares: Construct 1; green triangles: Construct 3; purple triangles: Construct 5. (A) Remaining target cells; (B)

FIG. 14: Testing the dnTGFβRII module. Blue circles: media only; red circles: +10 ng/ml rhTGF-β.

FIG. 15: Testing the dSHP2 module. Blue circles: non-transduced SupT1 cells; CD19+ SupT1 cells; CD19+PDL+ SupT1 cells.

FIG. 16: Re-stimulation assay. Red bars: target cells; blue bars: T cells. Top row: CD19+ SupT1 cells; bottom row: CD22+ SupT1 cells.

FIG. 17: Identification of a sub-optimal dose of cells expressing Construct 1 to serve as a starting point for Construct 5 comparison.

FIG. 18: In vivo comparison of Construct 1, 3, and 5. Cells expressing Construct 1 are unable to control tumour burden at this dosage level (2.5×10⁶ T cells). Cells expressing Construct 3 or Construct 5 show improved activity. Construct 5 in particular shows control of tumour burden to day 23 in all mice. The difference in flux is statistically significant compared to Construct 1.

FIG. 19: In vivo comparison of Construct 1, 3, and 5 in CD19 knock-out Nalm6 mice. Cells expressing Construct 1 are unable to control tumour burden at this dosage level (2.5×10⁶ T cells). Cells expressing Construct 3 or Construct 5 show improved activity. Construct 5 in particular shows control of tumour burden to day 27 in all but one mice.

### DETAILED DESCRIPTION

### Chimeric Antigen Receptors (CARs)

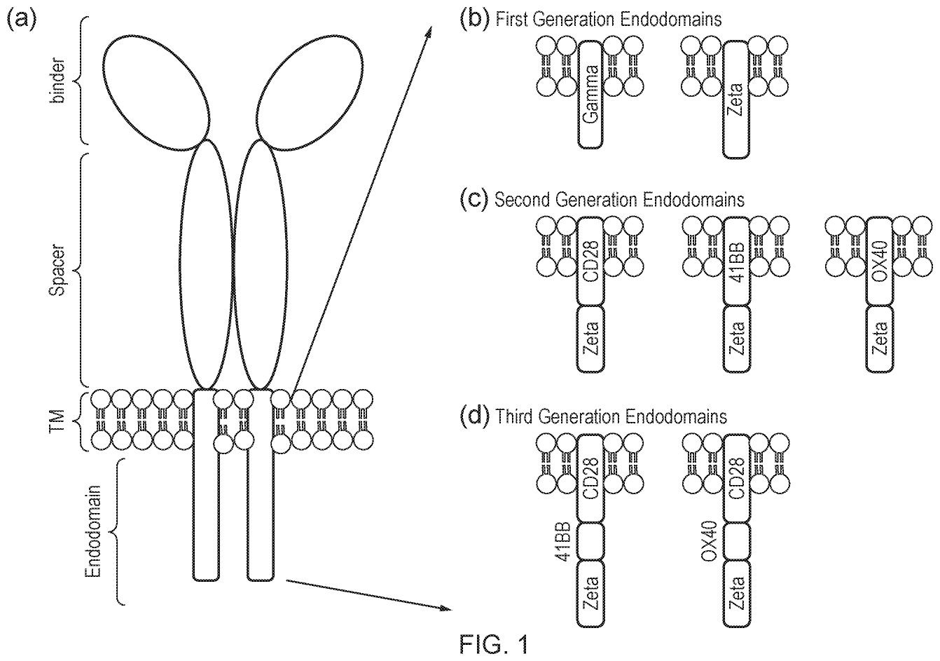

CARs, which are shown schematically in FIG. 1, are chimeric type I trans-membrane proteins which connect an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like antigen binding site. A spacer domain is usually necessary to isolate the binder from the membrane and to allow it a suitable orientation. A common spacer domain used is the Fc of IgG1. More compact spacers can suffice e.g. the stalk from CD8α and even just the IgG1 hinge alone, depending on the antigen. A trans-membrane domain anchors the protein in the cell membrane and connects the spacer to the endodomain.

Early CAR designs had endodomains derived from the intracellular parts of either the γ chain of the FcεR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3ζ results in second generation receptors which can transmit an activating and co-stimulatory signal simultaneously after antigen recognition. The co-stimulatory domain most commonly used is that of CD28. This supplies the most potent co-stimulatory signal-namely immunological signal 2, which triggers T-cell proliferation. Some receptors have also been described which include TNF receptor family endodomains, such as the closely related OX40 and 41BB which transmit survival signals. Even more potent third generation CARs have now been described which have endodomains capable of transmitting activation, proliferation and survival signals.

CAR-encoding nucleic acids may be transferred to T cells using, for example, retroviral vectors. Lentiviral vectors may be employed. In this way, a large number of cancer-specific T cells can be generated for adoptive cell transfer. When the CAR binds the target-antigen, this results in the transmission of an activating signal to the T-cell it is expressed on. Thus the CAR directs the specificity and cytotoxicity of the T cell towards tumour cells expressing the targeted antigen.

The first aspect of the invention relates to a cell which co-expresses a first CAR and a second CAR, wherein one CAR binds CD19 and the other CAR binds CD22, such that a T-cell can recognize a target cells expressing either of these markers.

Thus, the antigen binding domains of the first and second CARs of the present invention bind to different antigens and both CARs comprise an activating endodomain. In addition, each CAR uses a different intracellular signalling domain. The two CARs may comprise spacer domains which may be the same, or sufficiently different to prevent cross-pairing of the two different receptors. A cell can hence be engineered to activate upon recognition of either or both CD19 and CD22. This is useful in the field of oncology as indicated by the Goldie-Coldman hypothesis: sole targeting of a single antigen may result in tumour escape by modulation of said antigen due to the high mutation rate inherent in most cancers. By simultaneously targeting two antigens, the probably of such escape is exponentially reduced.

It is important that the two CARs do not heterodimerize.

The first and second CAR of the T cell of the present invention may be produced as a polypeptide comprising both CARs, together with a cleavage site.

### Signal Peptide

The CARs of the cell of the present invention may comprise a signal peptide so that when the CAR is expressed inside a cell, such as a T-cell, the nascent protein is directed to the endoplasmic reticulum and subsequently to the cell surface, where it is expressed.

The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

The signal peptide may be at the amino terminus of the molecule.

The signal peptide may comprise the SEQ ID No. 27, 28 or 29 or a variant thereof having 5, 4, 3, 2 or 1 amino acid mutations (insertions, substitutions or additions) provided that the signal peptide still functions to cause cell surface expression of the CAR.

### SEQ ID No. 27: MGTSLLCWMALCLLGADHADG

The signal peptide of SEQ ID No. 27 is compact and highly efficient. It is predicted to give about 95% cleavage after the terminal glycine, giving efficient removal by signal peptidase.

### SEQ ID No. 28: MSLPVTALLLPLALLLHAARP

The signal peptide of SEQ ID No. 28 is derived from IgG1.

### SEQ ID No. 29: MAVPTQVLGLLLLWLTDARC

The signal peptide of SEQ ID No. 29 is derived from CD8.

The signal peptide for the first CAR may have a different sequence from the signal peptide of the second CAR.

### CD19

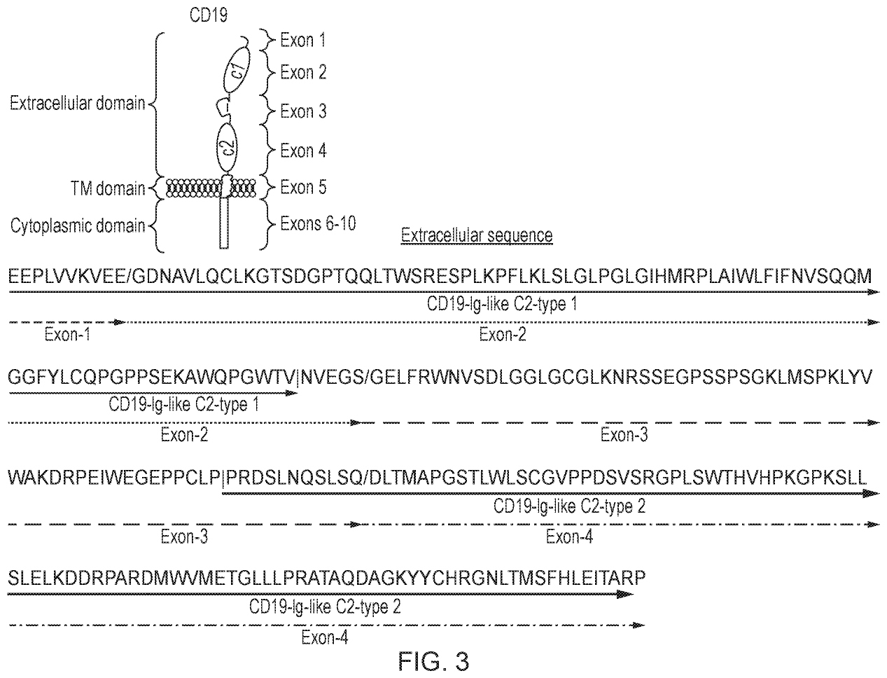

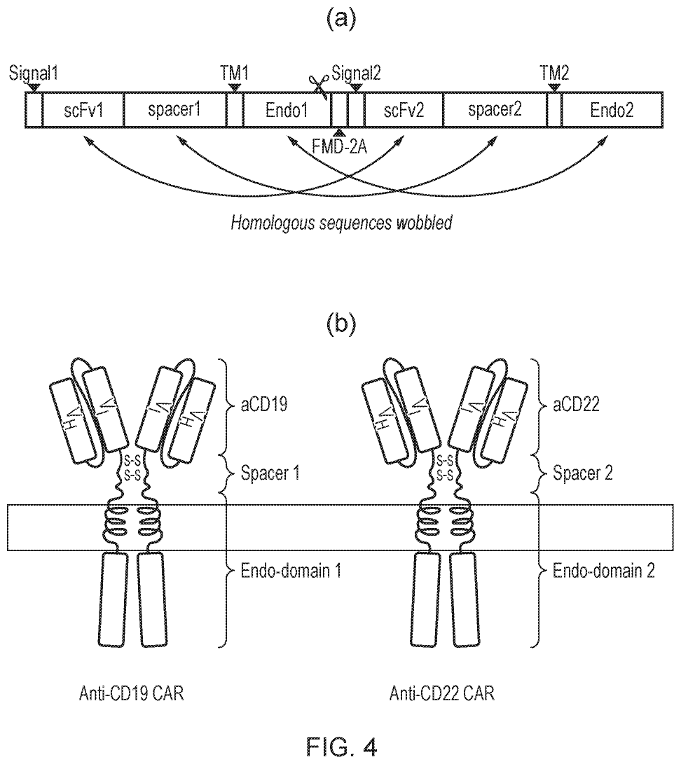

The human CD19 antigen is a 95 kDa transmembrane glycoprotein belonging to the immunoglobulin superfamily. CD19 is classified as a type I transmembrane protein, with a single transmembrane domain, a cytoplasmic C-terminus, and extracellular N-terminus. The general structure for CD19 is illustrated in FIG. 3.

CD19 is a biomarker for normal and neoplastic B cells, as well as follicular dendritic cells. In fact, it is present on B cells from earliest recognizable B-lineage cells during development to B-cell blasts but is lost on maturation to plasma cells. It primarily acts as a B cell co-receptor in conjunction with CD21 and CD81. Upon activation, the cytoplasmic tail of CD19 becomes phosphorylated, which leads to binding by Src-family kinases and recruitment of PI-3 kinase. CD19 is expressed very early in B-cell differentiation and is only lost at terminal B-cell differentiation into plasma cells. Consequently, CD19 is expressed on all B-cell malignancies apart from multiple myeloma.

Different designs of CARs have been tested against CD19 in different centres, as outlined in the following Table:

TABLE 1

| Centre | Binder | Endodomain |
|---|---|---|
| University College London | Fmc63 | CD3-Zeta |
| Memorial Sloane Kettering | SJ25C1 | CD28-Zeta |
| NCI/KITE | Fmc63 | CD28-Zeta |
| Baylor, Centre for Cell and | Fmc63 | CD3-Zeta/ |
| Gene Therapy | | CD28-Zeta |
| UPENN/Novartis | Fmc63 | 41BB-Zeta |
| University College London | CAT19 | 41BB-Zeta |

As shown above, most of the studies conducted to date have used an scFv derived from the hybridoma fmc63 as part of the binding domain to recognize CD19.

As shown in FIG. 3, the gene encoding CD19 comprises ten exons: exons 1 to 4 encode the extracellular domain; exon 5 encodes the transmembrane domain; and exons 6 to 10 encode the cytoplasmic domain,

In the CD19/CD22 OR gate of the present invention, the antigen-binding domain of the anti-CD19 CAR may bind an epitope of CD19 encoded by exon 1 of the CD19 gene.

In the CD19/CD22 OR gate of the present invention, the antigen-binding domain of the anti-CD19 CAR may bind an epitope of CD19 encoded by exon 3 of the CD19 gene.

In the CD19/CD22 OR gate of the present invention, the antigen-binding domain of the anti-CD19 CAR may bind an epitope of CD19 encoded by exon 4 of the CD19 gene.

The present inventors have developed an anti-CD19 CAR which has improved properties compared to a known anti-CD19 CAR which comprises the binder fmc63 (see WO2016/102965, Examples 2 and 3, the content of which are hereby incorporated by reference). The antigen binding domain of the CAR is based on the CD19 binder CD19ALAb, which has the CDRs and VH/VL regions identified below.

The present disclosure therefore also provides a CAR which comprises a CD19-binding domain which comprises a) a heavy chain variable region (VH) having complementarity determining regions (CDRs) with the following sequences:

CDR1-
(SEQ ID No. 1)
SYWMN;
CDR2-
(SEQ ID NO. 2)
QIWPGDGDTNYNGKFK
CDR3-
(SEQ ID No. 3)
RETTTVGRYYYAMDY;

and
b) a light chain variable region (VL) having CDRs with the following sequences:

CDR1-
(SEQ ID No. 4)
KASQSVDYDGDSYLN;
CDR2-
(SEQ ID No. 5)
DASNLVS
CDR3-
(SEQ ID NO. 6)
QQSTEDPWT.

It may be possible to introduce one or more mutations (substitutions, additions or deletions) into the or each CDR without negatively affecting CD19-binding activity. Each CDR may, for example, have one, two or three amino acid mutations.

The CAR of the present disclosure may comprise one of the following amino acid sequences:

(Murine CD19ALAb scFv sequence)
SEQ ID No. 12
QVQLQQSGAELVRPGSSVKISCKASGYAFSSYWMNWVKQRPGQGLEWIG
QIWPGDGDTNYNGKFKGKATLTADESSSTAYMQLSSLASEDSAVYFCAR
RETTTVGRYYYAMDYWGQGTTVTVSSDIQLTQSPASLAVSLGQRATISC
KASQSVDYDGDSYLNWYQQIPGQPPKLLIYDASNLVSGIPPRFSGSGSG
TDFTLNIHPVEKVDAATYHCQQSTEDPWTFGGGTKLEIK
(Humanised CD19ALAb scFv sequence-Heavy 19,
Kappa 16)
SEQ ID No. 13
QVQLVQSGAEVKKPGASVKLSCKASGYAFSSYWMNWVRQAPGQSLEWIG
QIWPGDGDTNYNGKFKGRATLTADESARTAYMELSSLRSGDTAVYFCAR
RETTTVGRYYYAMDYWGKGTLVTVSSDIQLTQSPDSLAVSLGERATINC
KASQSVDYDGDSYLNWYQQKPGQPPKLLIYDASNLVSGVPDRFSGSGSG
TDFTLTISSLQAADVAVYHCQQSTEDPWTFGQGTKVEIKR
(Humanised CD19ALAb scFv sequence Heavy 19,
Kappa 7)
SEQ ID No. 14
QVQLVQSGAEVKKPGASVKLSCKASGYAFSSYWMNWVRQAPGQSLEWIG
QIWPGDGDTNYNGKFKGRATLTADESARTAYMELSSLRSGDTAVYFCAR
RETTTVGRYYYAMDYWGKGTLVTVSSDIQLTQSPDSLAVSLGERATINC
KASQSVDYDGDSYLNWYQQKPGQPPKVLIYDASNLVSGVPDRFSGSGSG
TDFTLTISSLQAADVAVYYCQQSTEDPWTFGQGTKVEIKR

The scFv may be in a VH-VL orientation (as shown in SEQ ID Nos. 12, 13 and 14) or a VL-VH orientation.

The CAR of the present disclosure may comprise one of the following VH sequences:

(Murine CD19ALAb VH sequence)
SEQ ID No. 7
QVQLQQSGAELVRPGSSVKISCKASGYAFSSYWMNWVKQRPGQGLEWIG
QIWPGDGDTNYNGKFKGKATLTADESSSTAYMQLSSLASEDSAVYFCAR
RETTTVGRYYYAMDYWGQGTTVTVSS
(Humanised CD19ALAb VH sequence)
SEQ ID No. 8
QVQLVQSGAEVKKPGASVKLSCKASGYAFSSYWMNWVRQAPGQSLEWIG
QIWPGDGDTNYNGKFKGRATLTADESARTAYMELSSLRSGDTAVYFCAR
RETTTVGRYYYAMDYWGKGTLVTVSS

The CAR of the present disclosure may comprise one of the following VL sequences:

(Murine CD19ALAb VL sequence)
SEQ ID No. 9
DIQLTQSPASLAVSLGQRATISCKASQSVDYDGDSYLNWYQQIPGQPPK
LLIYDASNLVSGIPPRFSGSGSGTDFTLNIHPVEKVDAATYHCQQSTED
PWTFGGGTKLEIK
(Humanised CD19ALAb VL sequence, Kappa 16)
SEQ ID No. 10
DIQLTQSPDSLAVSLGERATINCKASQSVDYDGDSYLNWYQQKPGQPPK
LLIYDASNLVSGVPDRFSGSGSGTDFTLTISSLQAADVAVYHCQQSTED
PWTFGQGTKVEIKR
(Humanised CD19ALAb VL sequence, Kappa 7)
SEQ ID No. 11
DIQLTQSPDSLAVSLGERATINCKASQSVDYDGDSYLNWYQQKPGQPPK
VLIYDASNLVSGVPDRFSGSGSGTDFTLTISSLQAADVAVYYCQQSTED
PWTFGQGTKVEIKR

The CAR of the present invention may comprise a CD19-binding domain which comprises a) a heavy chain variable region (VH) having complementarity determining regions (CDRs) with the following sequences:

CDR1-
(SEQ ID No. 30)
GYAFSSS;
CDR2-
(SEQ ID NO. 31)
YPGDED
CDR3-
(SEQ ID No. 32)
SLLYGDYLDY;

and
b) a light chain variable region (VL) having CDRs with the following sequences:

CDR1-
(SEQ ID No. 33)
SASSSVSYMH;
CDR2-
(SEQ ID No. 34)
DTSKLAS
CDR3-
(SEQ ID NO. 35)
QQWNINPLT.

The CD19 binding domain may comprise the 6 CDRs defined above grafted on to a human antibody framework.

The CD19 binding domain may comprise a VH domain having the sequence shown as SEQ ID No. 36 and/or or a VL domain having the sequence shown as SEQ ID No 37 or a variant thereof having at least 95% sequence identity.

(CAT19 VH)
SEQ ID No. 36
QVQLQQSGPELVKPGASVKISCKASGYAFSSSWMNWVKQRPGKGLEWIG
RIYPGDEDTNYSGKFKDKATLTADKSSTTAYMQLSSLTSEDSAVYFCAR
SLLYGDYLDYWGQGTTLTVSS
(CAT19 VL)
SEQ ID No. 37
QIVLTQSPAIMSASPGEKVTMTCSASSSVSYMHWYQQKSGTSPKRWIYD
TSKLASGVPDRFSGSGSGTSYFLTINNMEAEDAATYYCQQWNINPLTFG
AGTKLELKR

The CD19 binding domain may comprise an scFv in the orientation VH-VL.

The CD19 binding domain may comprise the sequence shown as SEQ ID No 38 or a variant thereof having at least 90% sequence identity.

(CAT19 scFv)
SEQ ID No. 38
QVQLQQSGPELVKPGASVKISCKASGYAFSSSWMNWVKQRPGKGLEWIG
RIYPGDEDTNYSGKFKDKATLTADKSSTTAYMQLSSLTSEDSAVYFCAR
SLLYGDYLDYWGQGTTLTVSSGGGGSGGGGSGGGGSQIVLTQSPAIMSA
SPGEKVTMTCSASSSVSYMHWYQQKSGTSPKRWIYDTSKLASGVPDRFS
GSGSGTSYFLTINNMEAEDAATYYCQQWNINPLTFGAGTKLELKR

The CAR of the disclosure may comprise a variant of the sequence shown as SEQ ID No. 21, 13, 7, 8, 9, 10, 14, 11, 26, 37, or 38 having at least 80, 85, 90, 95, 98 or 99% sequence identity, provided that the variant sequence retain the capacity to bind CD19 (when in conjunction with a complementary VL or VH domain, if appropriate).

The percentage identity between two polypeptide sequences may be readily determined by programs such as BLAST which is freely available at http://blast.ncbi.nlm.nih.gov.

### CD22

The human CD22 antigen is a molecule belonging to the SIGLEC family of lectins. It is found on the surface of mature B cells and on some immature B cells. Generally speaking, CD22 is a regulatory molecule that prevents the overactivation of the immune system and the development of autoimmune diseases.

CD22 is a sugar binding transmembrane protein, which specifically binds sialic acid with an immunoglobulin (Ig) domain located at its N-terminus. The presence of Ig domains makes CD22 a member of the immunoglobulin superfamily. CD22 functions as an inhibitory receptor for B cell receptor (BCR) signaling.

CD22 is a molecule of the IgSF which may exist in two isoforms, one with seven domains and an intra-cytoplasmic tail comprising of three ITIMs (immune receptor tyrosine-based inhibitory motifs) and an ITAM; and a splicing variant which instead comprises of five extracellular domains and an intra-cytoplasmic tail carrying one ITIM. CD22 is thought to be an inhibitory receptor involved in the control of B-cell responses to antigen. Like CD19, CD22 is widely considered to be a pan-B antigen, although expression on some non-lymphoid tissue has been described. Targeting of CD22 with therapeutic monoclonal antibodies and immunoconjugates has entered clinical testing.

Examples of anti-CD22 CARs are described by Haso et al. (Blood; 2013; 121 (7)). Specifically, anti-CD22 CARs with antigen-binding domains derived from m971, HA22 and BL22 scFvs are described.

The antigen-binding domain of the anti-CD22 CAR may bind CD22 with a K_{D} in the range 30-50 nM, for example 30-40 nM. The K_{D} may be about 32 nM.

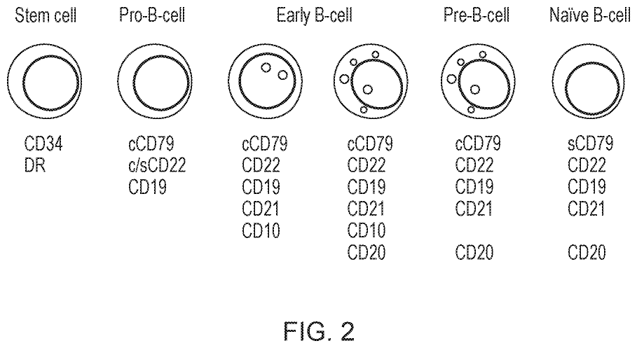

CD-22 has seven extracellular IgG-like domains, which are commonly identified as Ig domain 1 to Ig domain 7, with Ig domain 7 being most proximal to the B cell membrane and Ig domain 7 being the most distal from the Ig cell membrane (see Haso et al 2013 as above FIG. 2B).

The positions of the Ig domains in terms of the amino acid sequence of CD22 (http://www.uniprot.org/uniprot/P20273) are summarised in the following table:

| Ig domain | Amino acids |
|---|---|
| 7 | 20-138 |
| 6 | 143-235 |
| 5 | 242-326 |
| 4 | 331-416 |
| 3 | 419-500 |
| 2 | 505-582 |
| 1 | 593-676 |

The antigen-binding domain of the second CAR may bind to a membrane-distal epitope on CD22. The antigen-binding domain of the second CAR may bind to an epitope on Ig domain 7, 6, 5 or 4 of CD22, for example on Ig domain 5 of CD22. The antigen-binding domain of the second CAR may bind to an epitope located between amino acids 20-416 of CD22, for example between amino acids 242-326 of CD22.

The anti-CD22 antibodies HA22 and BL22 (Haso et al 2013 as above) and CD22ALAb, described below, bind to an epitope on Ig domain 5 of CD22.

The antigen binding domain of the second CAR may not bind to a membrane-proximal epitope on CD22. The antigen-binding domain of the second CAR may not bind to an epitope on Ig domain 3, 2 or 1 of CD22. The antigen-binding domain of the second CAR may not bind to an epitope located between amino acids 419-676 of CD22, such as between 505-676 of CD22.

The present inventors have developed an anti-CD22 CAR which has improved properties compared to a known anti-CD22 CAR which comprises the binder m971 (see WO2016/102965 Examples 2 and 3 and Haso et al (2013) as above, the contents of which are hereby incorporated by reference). The antigen binding domain of the CAR is based on the CD22 binder CD22ALAb, which has the CDRs and VH/VL regions identified below.

The present disclosure therefore also provides a CAR which comprises a CD22-binding domain which comprises
a) a heavy chain variable region (VH) having complementarity determining regions (CDRs) with the following sequences:

CDR1-
(SEQ ID No. 15)
NYWIN;
CDR2-
(SEQ ID No. 16)
NIYPSDSFTNYNQKFKD
CDR3-
(SEQ ID No. 17)
DTQERSWYFDV;

and
b) a light chain variable region (VL) having CDRs with the following sequences:

CDR1-
(SEQ ID No. 18)
RSSQSLVHSNGNTYLH;
CDR2-
(SEQ ID No. 19)
KVSNRFS
CDR3-
(SEQ ID NO. 20)
SQSTHVPWT.

It may be possible to introduce one or more mutations (substitutions, additions or deletions) into the or each CDR without negatively affecting CD22-binding activity. Each CDR may, for example, have one, two or three amino acid mutations.

The CAR of the present disclosure may comprise one of the following amino acid sequences:

(Murine CD22ALAb scFv sequence)
SEQ ID No. 25
QVQLQQPGAELVRPGASVKLSCKASGYTFTNYWINWVKQRPGQGLEWIG
NIYPSDSFTNYNQKFKDKATLTVDKSSSTAYMQLSSPTSEDSAVYYCTR
DTQERSWYFDVWGAGTTVTVSSDVVMTQTPLSLPVSLGDQASISCRSSQ
SLVHSNGNTYLHWYLQKPGQSPKLLIYKVSNRFSGVPDRFSGSGSGTDF
TLKISRVEAEDLGLYFCSQSTHVPWTFGGGTKLEIK
(Humanised CD22ALAb scFv sequence)
SEQ ID No. 26
EVQLVESGAEVKKPGSSVKVSCKASGYTFTNYWINWVRQAPGQGLEWIG
NIYPSDSFTNYNQKFKDRATLTVDKSTSTAYLELRNLRSDDTAVYYCTR
DTQERSWYFDVWGQGTLVTVSSDIVMTQSPATLSVSPGERATLSCRSSQ
SLVHSNGNTYLHWYQQKPGQAPRLLIYKVSNRFSGVPARFSGSGSGVEF
TLTISSLQSEDFAVYYCSQSTHVPWTFGQGTRLEIK

The scFv may be in a VH-VL orientation (as shown in SEQ ID Nos 25 and 26) or a VL-VH orientation.

The CAR of the present disclosure may comprise one of the following VH sequences:

(Murine CD22ALAb VH sequence)
SEQ ID No. 21
QVQLQQPGAELVRPGASVKLSCKASGYTFTNYWINWVKQRPGQGLEWIG
NIYPSDSFTNYNQKFKDKATLTVDKSSSTAYMQLSSPTSEDSAVYYCTR
DTQERSWYFDVWGAGTTVTVSS
(Humanised CD22ALAb VH sequence)
SEQ ID No. 22
EVQLVESGAEVKKPGSSVKVSCKASGYTFTNYWINWVRQAPGQGLEWIG
NIYPSDSFTNYNQKFKDRATLTVDKSTSTAYLELRNLRSDDTAVYYCTR
DTQERSWYFDVWGQGTLVTVSS

The CAR of the present disclosure may comprise one of the following VL sequences:

(Murine CD22ALAb VL sequence)
SEQ ID No. 23
DVVMTQTPLSLPVSLGDQASISCRSSQSLVHSNGNTYLHWYLQKPGQSP
KLLIYKVSNRFSGVPDRFSGSGSGTDFTLKISRVEAEDLGLYFCSQSTH
VPWTFGGGTKLEIK
(Humanised CD22ALAb VL sequence)
SEQ ID No. 24
DIVMTQSPATLSVSPGERATLSCRSSQSLVHSNGNTYLHWYQQKPGQAP
RLLIYKVSNRFSGVPARFSGSGSGVEFTLTISSLQSEDFAVYYCSQSTH
VPWTFGQGTRLEIK

The CAR of the disclosure may comprise a variant of the sequence shown as SEQ ID No. 25, 26, 21, 22, 23 or 24 having at least 80, 85, 90, 95, 98 or 99% sequence identity, provided that the variant sequence retain the capacity to bind CD22 (when in conjunction with a complementary VL or VH domain, if appropriate).

Other anti-CD22 antibodies are known, such as the mouse anti-human CD22 antibodies 1D9-3, 3B4-13, 7G6-6, 6C4-6, 4D9-12, 5H4-9, 10C1-D9, 15G7-2, 2B12-8, 2C4-4 and 3E10-7; and the humanised anti-human CD22 antibodies LT22 and Inotuzumab (G5_44). Table 1 summarises the, VH, VL and CDR sequences (in bold and underlined) and the position of the target epitope on CD22 for each antibody. These antibodies (or their CDR sequences) are suitable for use in the CD22 CAR of the present invention.

TABLE 1

| | | | Position |
|---|---|---|---|
| | | | of |
| | | | epitope |
| Antibody | VH | VL | on CD22 |
| 1D9-3 | EVOLVESGGGLVQPKGSLK | DIVMTQSQKFMSTSVGD | Domain 1 |
| | LSCAASGFTFNTYAMHWVR | RVSITCKASQNVRTAVA | and 2 |
| | QAPGKGLEWVARIRSKSSN | WYQQKPGQSPKALIYLA | |
| | YATYYADSVKDRFTISRDD | SNRHTGVPDRFTGSGSG | |
| | SQSMLYLQMNNLKTEDTAM | TDFTLTISNVQSEDLADY | |
| | YYCVVDYLYAMDYWGQGT | FCLQHWNYPFTFGSGTK | |
| | SVTVSS | LEIK | |
| | (SEQ ID No. 39) | (SEQ ID No. 40) | |
| 3B4-13 | QVQLQQSGAELVRPGASVT | QAVVTQESALTTSPGET | Domain 1 |
| | LSCKASGYTFTDYEMHWVK | VTLTCRSSAGAVTTSNY | |
| | QTPVHGLEWIGAIDPETGA | ANWVQEKPDHLFTGLIG | |
| | TAYNQKFKGKAILTADKSSS | GTNNRAPGVPARFSGSL | |
| | TAYMDLRSLTSEDSAVYYC | IGDKAALTITGAQTEDEAI | and 2 |
| | TRYDYGSSPWFAYWGQGT | YFCALWNSNHWVFGGG | |
| | LVTVSA | TKLTVL | |
| | (SEQ ID No. 41) | (SEQ ID No. 42) | |
| 7G6-6 | QVQLQQPGAELVMPGASV | DIVMSQSPSSLAVSVGE | Domain 1 |
| | KLSCKASGYTFTSYWMHW | KVTMSCKSSQSLLYSSN | and 2 |
| | VKQRPGQGLEWIGEIDPSD | QKNYLAWYQQKPGQSP | |
| | SYTNYNQKFKGKATLTVDK | KLLIYWASTRESGVPDRF | |
| | SSSTAYMQLSSLTSEDSAV | TGSGSGTDFTLTISSVKA | |
| | YYCARGYYGSSSFDYWGQ | EDLAVYYCQQYYSYTFG | |
| | GTTLTVSS | GGTKLEIK | |
| | (SEQ ID No. 43) | (SEQ ID No. 44) | |
| 6C4-6 | QVQLKESGPGLVAPSQSLSI | DIQMTQSPASLSASVGE | Domain 3 |
| | TCTVSGFSLTSYGVHWVRQ | TVTITCRASENIYSYLAW | |
| | PPGKGLEWLVVIWSDGSTT | YQQKQGKSPQLLVYNAK | |
| | YNSALKSRLSISKDNSKSQ | TLAEGVPSRFSGSGSGT | |
| | VFLKMNSLQTDDTAMYYCA | QFSLKINSLQPEDFGSYY | |
| | RHADDYGFAWFAYWGQG | CQHHYGTPPTFGGGTKL | |
| | TLVTVSA | EIK | |
| | (SEQ ID No. 45) | (SEQ ID No. 46) | |
| 4D9-12 | EFQLQQSGPELVKPGASVK | DIQMTQSPSSLSASLGE | Domain 4 |
| | ISCKASGYSFTDYNMNWVK | RVSLTCRASQEISGYLS | |
| | QSNGKSLEWIGVINPNYGT | WLQQKPDGTIKRLIYAAS | |
| | TSYNQKFKGKATLTVDQSS | TLDSGVPKRFSGSRSGS | |
| | STAYMQLNSLTSEDSAVYY | DYSLTISSLESEDFADYY | |
| | CARSSTTVVDWYFDVWGT | CLQYASYPFTFGSGTKL | |
| | GTTVTVSS | EIK | |
| | (SEQ ID No. 47) | (SEQ ID No. 48) | |
| 5H4-9 | QVQVQQPGAELVRPGTSV | DVVMTQTPLSLPVSLGD | Domain 4 |
| | KLSCKASGYTFTRYWMYW | QASISCRSSQSLVHSNG | |
| | VKQRPGQGLEWIGVIDPSD | NTYLHWYLQKPGQSPKL | |
| | NFTYYNQKFKGKATLTVDT | LIYKVSNRFSGVPDRFSG | |
| | SSSTAYMQLSSLTSEDSAV | SGSGTDFTLKISRVEAED | |
| | YYCARGYGSSYVGYWGQG | LGVYFCSQSTHVPPWTF | |
| | TTLTVSS | GGGTKLEIK | |
| | (SEQ ID No. 49) | (SEQ ID No. 50) | |
| 10C1-D9 | QVTLKESGPGILQSSQTLSL | DIQMTQTTSSLSASLGDR | Domain 4 |
| | TCSFSGFSLSTSDMGVSWI | VTISCRASQDISNYLNWY | |
| | RQPSGKGLEWLAHIYWDD | QQKPDGTVKLLIYYTSRL | |
| | DKRYNPSLKSRLTISKDASR | HSGVPSRFSGSGSGTDY | |
| | NQVFLKIATVDTADTATYYC | SLTISNLEQEDIATYFCQ | |
| | ARSPWIYYGHYWCFDVWG | QGNTLPFTFGSGTKLEIK | |
| | TGTTVTVSS | (SEQ ID No. 52) | |
| | (SEQ ID No. 51) | | |
| 15G7-2 | QVQLQQSGAELVKPGASVK | QIVLTQSPAIMSASPGEK | Domain 4 |
| | LSCKASGYTFTEYTIHWVK | VTMTCSASSSVSYMYW | |
| | QRSGQGLEWIGWFYPGSG | YQQKPGSSPRLLIYDTSN | |
| | SIKYNEKFKDKATLTADKSS | LASGVPVRFSGSGSGTS | |
| | STVYMELSRLTSEDSAVYF | YSLTISRMEAEDAATYYC | |
| | CARHGDGYYLPPYYFDYW | QQWSSYPLTFGAGTKLE | |
| | GQGTTLTVSS | LK | |
| | (SEQ ID No. 53) | (SEQ ID No. 54) | |
| 2B12-8 | QVQLQQSGAELARPGASVK | DIVLTQSPATLSVTPGDS | Domain 4 |
| | LSCKASGYIFTSYGISWVKQ | VSLSCRASQSISTNLHW | |
| | RTGQGLEWIGEIYPRSGNT | YQQKSHASPRLLIKYASQ | |
| | YYNEKFKGKATLTADKSSS | SVSGIPSRFSGSGSGTD | |
| | TAYMELRSLTSEDSAVYFC | FTLSINSVETEDFGIFFCQ | |
| | ARPIYYGSREGFDYWGQGT | QSYSWPYTFGGGTKLEI | |
| | TLTVSS | K | |
| | (SEQ ID No. 55) | (SEQ ID No. 56) | |
| 2C4-4 | QVQLQQPGAELVMPGASV | DVLMTQTPLSLPVSLGD | Domain 5- |
| | KLSCKASGYTFTSYWMHW | QASISCRSSQSIVHSNGN | 7 |
| | VKQRPGQGLEWIGEIDPSD | TYLEWYLQKPGQSPKLLI | |
| | SYTNYNQKFKGKSTLTVDK | YKVSNRFSGVPDRFSGS | |
| | SSSTAYIQLSSLTSEDSAVY | ESGTDFTLKISRVEAEDL | |
| | YCARWASYRGYAMDYWG | GVYYCFQGSHVPWTFG | |
| | QGTSVTVSS | GGTKLEIK | |
| | (SEQ ID No. 57) | (SEQ ID No. 58) | |
| 3E10-7 | EFQLQQSGPELVKPGASVK | DIQMTQSPSSLSASLGE | Domain 5- |
| | ISCKASGYSFTDYNMNWVK | RVSLTCRASQEISGYLS | 7 |
| | QSNGKSLEWIGVINPNYGT | WLQQKPDGTIKRLIYAAS | |
| | TSYNQRFKGKATLTVDQSS | TLDSGVPKRFSGSRSGS | |
| | STAYMQLNSLTSEDSAVYY | DYSLTISSLESEDFADYY | |
| | CARSGLRYWYFDVWGTGT | CLQYASYPFTFGSGTKL | |
| | TVTVSS | EIK | |
| | (SEQ ID No. 59) | (SEQ ID No. 60) | |
| Inotuzumab | EVQLVQSGAEVKKPGASVK | DVQVTQSPSSLSASVGD | Domain 7 |
| G5_44 | VSCKASGYRFTNYWIHWVR | RVTITCRSSQSLANSYG | |
| | QAPGQGLEWIGGINPGNNY | NTFLSWYLHKPGKAPQL | |
| | ATYRRKFQGRVTMTADTST | LIYGISNRFSGVPDRFSG | |
| | STVYMELSSLRSEDTAVYY | SGSGTDFTLTISSLOPED | |
| | CTREGYGNYGAWFAYWG | FATYYCLQGTHQPYTFG | |
| | QGTLVTVSS | QGTKVEIKR | |
| | (SEQ ID No. 61) | (SEQ ID No. 62) | |
| 9A8-1 | EVOLVESGGGLVQPGRSLK | DIQMTQSPSSLSASLGD | Domains |
| | LSCAASGFTFSNFAMAWVR | RVTITCRSSQDIGNYLTW | 1 and 2 |
| | QPPTKGLEWVASISTGGGN | FQQKVGRSPRRMIYGAI | |
| | TYYRDSVKGRFTISRDDAK | KLEDGVPSRFSGSRSGS | |
| | NTQYLQMDSLRSEDTATYY | DYSLTISSLESEDVADYQ | |
| | CARQRNYYDGSYDYEGYT | CLQSIQYPFTFGSGTKLE | |
| | MDAWGQGTSVTVSS (SEQ | IK (SEQ ID No. 64) | |
| | ID No. 63) | | |

The present disclosure also provides a CAR which comprises a CD22-binding domain which comprises
a) a heavy chain variable region (VH) having complementarity determining regions (CDRs) with the following sequences:

CDR1-
(SEQ ID No. 101)
NFAMA;
CDR2-
(SEQ ID No. 102)
SISTGGGNTYYRDSVKG
CDR3-
(SEQ ID No. 103)
QRNYYDGSYDYEGYTMDA;

and
b) a light number chain variable region (VL) having CDRs with the following sequences:

CDR1-
(SEQ ID No. 104)
RSSQDIGNYLT;
CDR2-
(SEQ ID No. 105)
GAIKLED
CDR3-
(SEQ ID No. 106)
LQSIQYP.

It may be possible to introduce one or more mutations (substitutions, additions or deletions) into the or each CDR without negatively affecting CD22-binding activity. Each CDR may, for example, have one, two or three amino acid mutations.

The CAR of the present disclosure may comprise the following VH sequences:

(9A8-1 VH sequence)
SEQ ID No. 63
EVQLVESGGGLVQPGRSLKLSCAASGFTFSNFAMAWVRQPPTKGLEWVA
SISTGGGNTYYRDSVKGRFTISRDDAKNTQYLQMDSLRSEDTATYYCAR
QRNYYDGSYDYEGYTMDAWGQGTSVTVSS

The CAR of the present disclosure may comprise the following VL sequences:

(9A8-1 VL sequence)
SEQ ID No. 64
DIQMTQSPSSLSASLGDRVTITCRSSQDIGNYLTWFQQKVGRSPRRMIY
GAIKLEDGVPSRFSGSRSGSDYSLTISSLESEDVADYQCLQSIQYPFTF
GSGTKLEIK

The scFv may be in a VH-VL orientation or a VL-VH orientation.

The CAR of the disclosure may comprise a variant of the sequence shown as SEQ ID No. 63 or 64 having at least 80, 85, 90, 95, 98 or 99% sequence identity, provided that the variant sequence retain the capacity to bind CD22 (when in conjunction with a complementary VL or VH domain, if appropriate).

### B-Cell Antigen Expression During B-Cell Ontogeny and Subsequent Tumours

CD19 is widely considered a pan-B antigen, although very occasionally, it may display some lineage infidelity. The CD19 molecule comprises of two extracellular IgSF domains separated by a smaller domain and a long intracytoplasmic tail, nearly as big as the extracellular portion of the molecule, carrying one ITAM. CD19 is a key molecule in the development and activation of B-cells. CD22 is a molecule of the IgSF which may exist in two isoforms, one with seven domains and an intra-cytoplasmic tail comprising of three ITIMs (immune receptor tyrosine-based inhibitory motifs) and an ITAM; and a splicing variant which instead comprises of five extracellular domains and an intra-cytoplasmic tail carrying one ITIM. CD22 is thought to be an inhibitory receptor involved in the control of B-cell responses to antigen. Like CD19, CD22 is widely considered to be a pan-B antigen, although expression on some non-lymphoid tissue has been described (Wen et al. (2012) J. Immunol. Baltim. Md 1950 188, 1075-1082). Targeting of CD22 with therapeutic monoclonal antibodies and immunoconjugates has entered clinical testing. Generation of CD22 specific CARs have been described (Haso et al, 2013, Blood: Volume 121; 7:1165-74, and James et al 2008, Journal of immunology, Volume 180; Issue 10; Pages 7028-38).

Detailed immunophentyping studies of B-cell leukaemias shows that while surface CD19 is always present, surface CD22 is almost always present. For instance, Raponi et al (2011, as above) studied the surface antigen phenotype of 427 cases of B-ALL and found CD22 present in 341 of cases studied.

The eventuality of CD19 down-regulation after CAR19 targeting described above may be explained by the Goldie-Coldman hypothesis. The Goldie-Coldman hypothesis predicts that tumor cells mutate to a resistant phenotype at a rate dependent on their intrinsic genetic instability and that the probability that a cancer would contain resistant clones depends on the mutation rate and the size of the tumor. While it may be difficult for cancer cells to become intrinsically resistant to the direct killing of cytotoxic T-cells, antigen loss remains possible. Indeed this phenomenon has been reported before with targeting melanoma antigens and EBV-driven lymphomas. According to Goldie-Coldman hypothesis, the best chance of cure would be to simultaneously attack non-cross resistant targets. Given that CD22 is expressed on nearly all cases of B-ALL, simultaneous CAR targeting of CD19 along with CD22 may reduce the emergence of resistant CD19 negative clones.

### Antigen Binding Domain

The antigen binding domain is the portion of the CAR which recognizes antigen. Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; a natural ligand of the target antigen; a peptide with sufficient affinity for the target; a single domain antibody; an artificial single binder such as a Darpin (designed ankyrin repeat protein); or a single-chain derived from a T-cell receptor.

The antigen binding domain of the CAR which binds to CD19 may be any domain which is capable of binding CD19. For example, the antigen binding domain may comprise a CD19 binder as described in Table 1.

The antigen binding domain of the CAR which binds to CD19 may comprise a sequence derived from one of the CD19 binders shown in Table 2.

TABLE 2

| Binder | References |
|---|---|
| HD63 | Pezzutto (Pezzutto, A. et al. J. Immunol. Baltim. Md |
| | 1950 138, 2793-2799 (1987) |
| 4g7 | Meeker et al (Meeker, T. C. et al. Hybridoma 3, |
| | 305-320 (1984) |
| Fmc63 | Nicholson et al (Nicholson, I. C. et al. Mol. |
| | Immunol. 34, 1157-1165 (1997) |
| B43 | Bejcek et al (Bejcek, B. E. et al. Cancer Res. 55, |
| | 2346-2351 (1995) |
| SJ25C1 | Bejcek et al (1995, as above) |
| BLY3 | Bejcek et al (1995, as above) |
| B4, or | Roguska et al (Roguska, M. A. et al. Protein Eng. 9, |
| re-surfaced, | |
| or humanized | |
| B4 | 895-904 (1996) |
| HB12b, | Kansas et al (Kansas, G. S. & Tedder, T. F. J. Immunol. |
| optimized and | Baltim. Md 1950 147, 4094-4102 (1991); Yazawa et al |
| humanized | (Yazawa et al Proc. Natl. Acad. Sci. U. S. A. 102, |
| | 15178-15183 (2005); Herbst et al (Herbst, R. et al. J. |
| | Pharmacol. Exp. Ther. 335, 213-222 (2010) |
| CAT19 | WO2016/139487 |
| CD19ALAb | WO2016/102965 |

The antigen binding domain of the CAR which binds to CD22 may be any domain which is capable of binding CD22. For example, the antigen binding domain may comprise a CD22 binder as described in Table 3.

TABLE 3

| Binder | References |
|---|---|
| M5/44 or | John et al (J. Immunol. Baltim. Md 1950 170, 3534- |
| humanized | 3543 (2003); and DiJoseph et al (Cancer Immunol. |
| M5/44 | Immunother. CII 54, 11-24 (2005) |
| M6/13 | DiJoseph et al (as above) |
| HD39 | Dorken et al (J. Immunol. Baltim. Md 1950 136, |
| | 4470-4479 (1986) |
| HD239 | Dorken et al (as above) |
| HD6 | Pezzutto et al (J. Immunol. Baltim. Md 1950 138, |
| | 98-103 (1987) |
| RFB-4, or | Campana et al (J. Immunol. Baltim. Md 1950 134, |
| humanized | 1524-1530 (1985); Krauss et al (Protein Eng. 16, 753 |
| RFB-4, or | 759 (2003), Kreitman et al (J. Clin. Oncol. Off. J. Am. |
| affinity matured | Soc. Clin. Oncol. 30, 1822-1828 (2012)) |
| To15 | Mason et al (Blood 69, 836-840 (1987)) |
| 4KB128 | Mason et al (as above) |
| S-HCL1 | Schwarting et al (Blood 65, 974-983 (1985)) |
| mLL2 (EPB-2), | Shih et al (Int. J. Cancer J. Int. Cancer 56, 538-545 |
| or humanized | (1994)), Leonard et al (J. Clin. Oncol. Off. J. Am. Soc. |
| mLL2-hLL2 | Clin. Oncol. 21, 3051-3059 (2003)) |
| M971 | Xiao et al (mAbs 1, 297-303 (2009)) |
| BC-8 | Engel et al (J. Exp. Med. 181, 1581-1586 (1995)) |
| HB22-12 | Engel et al (as above) |
| 9A8-1 | WO2019/220109 |
| CD22ALAb | WO2016/102965 |

### Spacer Domain

CARs comprise a spacer sequence to connect the antigen-binding domain with the transmembrane domain and spatially separate the antigen-binding domain from the endodomain. A flexible spacer allows the antigen-binding domain to orient in different directions to facilitate binding.

In the cell of the present invention, the first and second CARs may comprise different spacer molecules. For example, the spacer sequence may, for example, comprise an IgG1 Fc region, an IgG1 hinge or a human CD8 stalk or the mouse CD8 stalk. The spacer may alternatively comprise an alternative linker sequence which has similar length and/or domain spacing properties as an IgG1 Fc region, an IgG1 hinge or a CD8 stalk. A human IgG1 spacer may be altered to remove Fc binding motifs.

The spacer for the anti-CD19 CAR may comprise a CD8 stalk spacer, or a spacer having a length equivalent to a CD8 stalk spacer. The spacer for the anti-CD19 CAR may have at least 30 amino acids or at least 40 amino acids. It may have between 35-55 amino acids, for example between 40-50 amino acids. It may have about 46 amino acids.

The spacer for the anti-CD22 CAR may comprise an IgG1 hinge spacer, or a spacer having a length equivalent to an IgG1 hinge spacer. The spacer for the anti-CD22 CAR may have fewer than 30 amino acids or fewer than 25 amino acids. It may have between 15-25 amino acids, for example between 18-22 amino acids. It may have about 20 amino acids.

Examples of amino acid sequences for these spacers are given below:

(hinge-CH2CH3 of human IgG1)
SEQ ID No. 65
AEPKSPDKTHTCPPCPAPPVAGPSVFLFPPKPKDTLMIARTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGKKD
SEQ ID No. 66 (human CD8 stalk):
TTTPAPRPPTPAPTIASQPLSLRPEACRPAAGGAVHTRGLDFACDI
SEQ ID No. 67 (human IgG1 hinge):
AEPKSPDKTHTCPPCPKDPK
(CD2 ectodomain)
SEQ ID No. 68
KEITNALETWGALGQDINLDIPSFQMSDDIDDIKWEKTSDKKKIAQFRK
EKETFKEKDTYKLFKNGTLKIKHLKTDDQDIYKVSIYDTKGKNVLEKIF
DLKIQERVSKPKISWTCINTTLTCEVMNGTDPELNLYQDGKHLKLSQRV
ITHKWTTSLSAKFKCTAGNKVSKESSVEPVSCPEKGLD
(CD34 ectodomain)
SEQ ID No. 69
SLDNNGTATPELPTQGTFSNVSTNVSYQETTTPSTLGSTSLHPVSQHGN
EATTNITETTVKFTSTSVITSVYGNTNSSVQSQTSVISTVFTTPANVST
PETTLKPSLSPGNVSDLSTTSTSLATSPTKPYTSSSPILSDIKAEIKCS
GIREVKLTQGICLEQNKTSSCAEFKKDRGEGLARVLCGEEQADADAGAQ
VCSLLLAQSEVRPQCLLLVLANRTEISSKLQLMKKHQSDLKKLGILDFT
EQDVASHQSYSQKT

Since CARs are typically homodimers (see FIG. 1a), cross-pairing may result in a heterodimeric chimeric antigen receptor. This is undesirable for various reasons, for example: (1) the epitope may not be at the same “level” on the target cell so that a cross-paired CAR may only be able to bind to one antigen; (2) the VH and VL from the two different scFv could swap over and either fail to recognize target or worse recognize an unexpected and unpredicted antigen. The spacer of the first CAR may be sufficiently different from the spacer of the second CAR in order to avoid cross-pairing. The amino acid sequence of the first spacer may share less that 50%, 40%, 30% or 20% identity at the amino acid level with the second spacer.

### Coiled Coil Domain

CARs typically comprise a spacer sequence to connect the antigen-binding domain with the transmembrane domain. The spacer allows the antigen-binding domain to have a suitable orientation and reach. The spacer also provides segregation from phosphatases upon ligand engagement.

The CAR of the present invention may comprise a coiled coil spacer domain. In particular, the CAR specific for CD22 may comprise a coiled coil spacer domain. The coiled-coil spacer domain provides numerous advantages over the spacers previously described in the art.

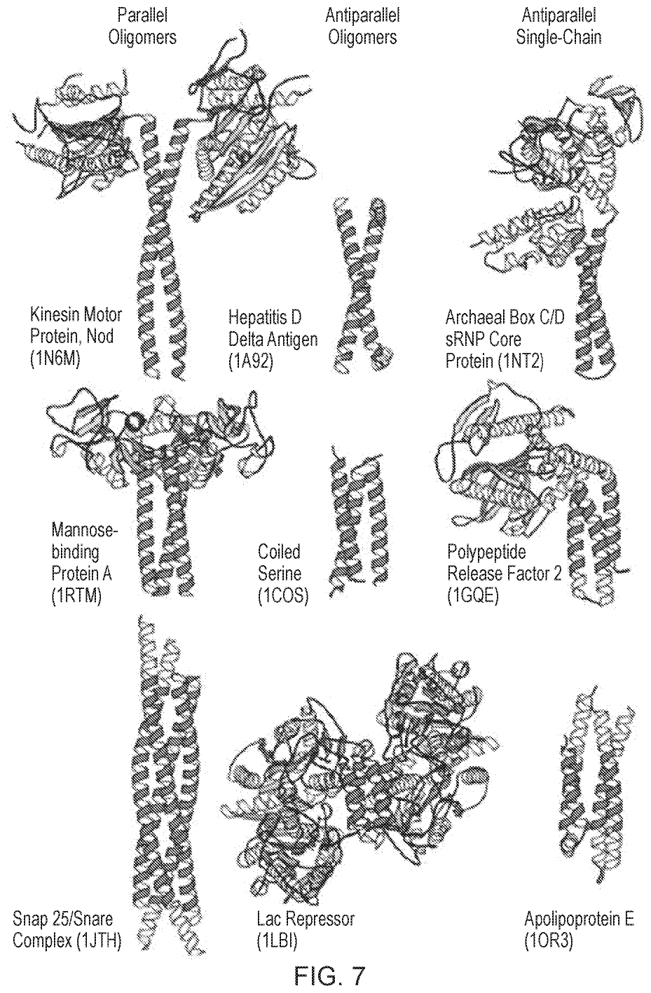

A coiled coil is a structural motif in which two to seven alpha-helices are wrapped together like the strands of a rope (FIG. 7). Many endogenous proteins incorporate coiled coil domains. The coiled coil domain may be involved in protein folding (e.g. it interacts with several alpha helical motifs within the same protein chain) or responsible for protein-protein interaction. In the latter case, the coiled coil can initiate homo or hetero oligomer structures.

As used herein, the terms ‘multimer’ and ‘multimerization’ are synonymous and interchangeable with ‘oligomer’ and ‘oligomerization’.

The structure of coiled coil domains is well known in the art. For example as described by Lupas & Gruber (Advances in Protein Chemistry; 2007; 70; 37-38).

Coiled coils usually contain a repeated pattern, hxxhcxc, of hydrophobic (h) and charged (c) amino-acid residues, referred to as a heptad repeat. The positions in the heptad repeat are usually labeled abcdefg, where a and d are the hydrophobic positions, often being occupied by isoleucine, leucine, or valine. Folding a sequence with this repeating pattern into an alpha-helical secondary structure causes the hydrophobic residues to be presented as a ‘stripe’ that coils gently around the helix in left-handed fashion, forming an amphipathic structure. The most favourable way for two such helices to arrange themselves in the cytoplasm is to wrap the hydrophobic strands against each other sandwiched between the hydrophilic amino acids. Thus, it is the burial of hydrophobic surfaces that provides the thermodynamic driving force for the oligomerization. The packing in a coiled-coil interface is exceptionally tight, with almost complete van der Waals contact between the side-chains of the a and d residues.

The α-helices may be parallel or anti-parallel, and usually adopt a left-handed super-coil. Although disfavoured, a few right-handed coiled coils have also been observed in nature and in designed proteins.

The coiled coil domain may be any coiled coil domain which is capable of forming a coiled coil multimer such that a complex of CARs or accessory polypeptides comprising the coiled coil domain is formed.

The relationship between the sequence and the final folded structure of a coiled coil domain are well understood in the art (Mahrenholz et al; Molecular & Cellular Proteomics; 2011; 10(5):M110.004994). As such the coiled coil domain may be a synthetically generated coiled coil domain.

Examples of proteins which contain a coiled coil domain include, but are not limited to, kinesin motor protein, hepatitis D delta antigen, archaeal box C/D sRNP core protein, cartilage-oligomeric matrix protein (COMP), mannose-binding protein A, coiled-coil serine-rich protein 1, polypeptide release factor 2, SNAP-25, SNARE, Lac repressor or apolipoprotein E.

The sequence of various coiled coil domains is shown below:

Kinesin motor protein: parallel homodimer
(SEQ ID No. 70)
MHAALSTEVVHLRQRTEELLRCNEQQAAELETCKEQLFQSNMERKELHN
TVMDLRGN
Hepatitis D delta antigen: parallel homodimer
(SEQ ID No. 71)
GREDILEQWVSGRKKLEELERDLRKLKKKIKKLEEDNPWLGNIKGIIGK
Y
Archaeal box C/D sRNP core protein: anti-parallel
heterodimer
(SEQ ID No. 72)
RYVVALVKALEEIDESINMLNEKLEDIRAVKESEITEKFEKKIRELREL
RRDVEREIEEVM
Mannose-binding protein A: parallel homotrimer
(SEQ ID No. 73)
AIEVKLANMEAEINTLKSKLELTNKLHAFSM
Coiled-coil serine-rich protein 1: parallel
homotrimer
(SEQ ID No. 74)
EWEALEKKLAALESKLQALEKKLEALEHG
Polypeptide release factor 2: anti-parallel
heterotrimer
Chain A:
(SEQ ID No. 75)
INPVNNRIQDLTERSDVLRGYLDY
Chain B:
(SEQ ID No. 76)
VVDTLDQMKQGLEDVSGLLELAVEADDEETFNEAVAELDALEEKLAQLE
FR
SNAP-25 and SNARE: parallel heterotetramer
Chain A:
(SEQ ID No. 77)
IETRHSEIIKLENSIRELHDMFMDMAMLVESQGEMIDRIEYNVEHAVDY
VE
Chain B:
(SEQ ID No. 78)
ALSEIETRHSEIIKLENSIRELHDMFMDMAMLVESQGEMIDRIEYNVEH
AVDYVERAVSDTKKAVKY
Chain C:
(SEQ ID No. 79)
ELEEMQRRADQLADESLESTRRMLQLVEESKDAGIRTLVMLDEQGEQLE
RIEEGMDQINKDMKEAEKNL
Chain D:
(SEQ ID No. 80)
IETRHSEIIKLENSIRELHDMFMDMAMLVESQGEMIDRIEYNVEHAVDY
VE
Lac repressor: parallel homotetramer
(SEQ ID No. 81)
SPRALADSLMQLARQVSRLE
Apolipoprotein E: anti-parallel heterotetramer
(SEQ ID No. 82)
SGQRWELALGRFWDYLRWVQTLSEQVQEELLSSQVTQELRALMDETMKE
LKAYKSELEEQLTARLSKELQAAQARLGADMEDVCGRLVQYRGEVQAML
GQSTEELRVRLASHLRKLRKRLLRDADDLQKRLAVYQA

The coiled coil domain is capable of oligomerization. In certain embodiments, the coiled coil domain may be capable of forming a trimer, a tetramer, a pentamer, a hexamer or a heptamer.

A coiled-coil domain is different from a leucine zipper. Leucine zippers are super-secondary structures that function as a dimerization domains. Their presence generates adhesion forces in parallel alpha helices. A single leucine zipper consists of multiple leucine residues at approximately 7-residue intervals, which forms an amphipathic alpha helix with a hydrophobic region running along one side. This hydrophobic region provides an area for dimerization, allowing the motifs to “zip” together. Leucine zippers are typically 20 to 40 amino acids in length, for example approximately 30 amino acids.

Leucine zippers are typically formed by two different sequences, for example an acidic leucine zipper heterodimerizes with a basic leucine zipper. An example of a leucine zipper is the docking domain (DDD1) and anchoring domain (AD1) which are described in more detail below.

Leucine zippers form dimers, whereas the coiled-coiled spacers of the present invention for multimers (trimers and above). Leucine zippers heterodimerise in the dimerization portion of the sequence, whereas coiled-coil domains homodimerise.

A hyper-sensitive CAR may be provided by increasing the valency of the CAR. In particular, the use of a coiled coil spacer domain which is capable of interacting to form a multimer comprising more than two coiled coil domains, and therefore more than two CARs, increases the sensitivity to targets expressing low density ligands due to increasing the number of ITAMs present and avidity of the oligomeric CAR complex.

Thus there is provided herein a CAR-forming polypeptide comprising a coiled coil spacer domain which enables the multimerization of at least three CAR-forming polypeptidess. In other words, the CAR comprises a coiled coil domain which is capable of forming a trimer, a tetramer, a pentamer, a hexamer or a heptamer of coiled coil domains.

Examples of coiled coil domains which are capable of forming multimers comprising more than two coiled coil domains include, but are not limited to, those from cartilage-oligomeric matrix protein (COMP), mannose-binding protein A, coiled-coil serine-rich protein 1, polypeptide release factor 2, SNAP-25, SNARE, Lac repressor or apolipoprotein E (see SEQ ID Nos. 70-82 above).

The coiled coil domain may be the COMP coiled coil domain.

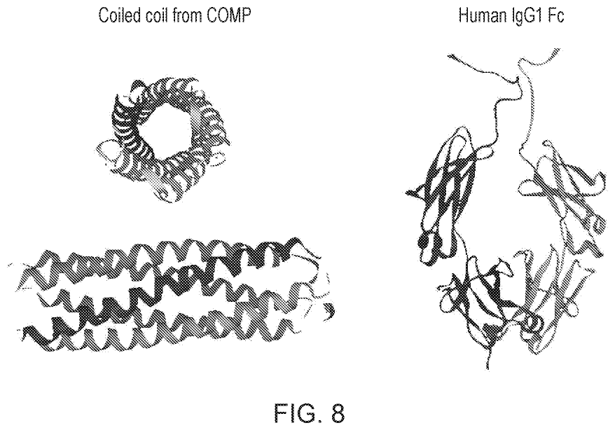

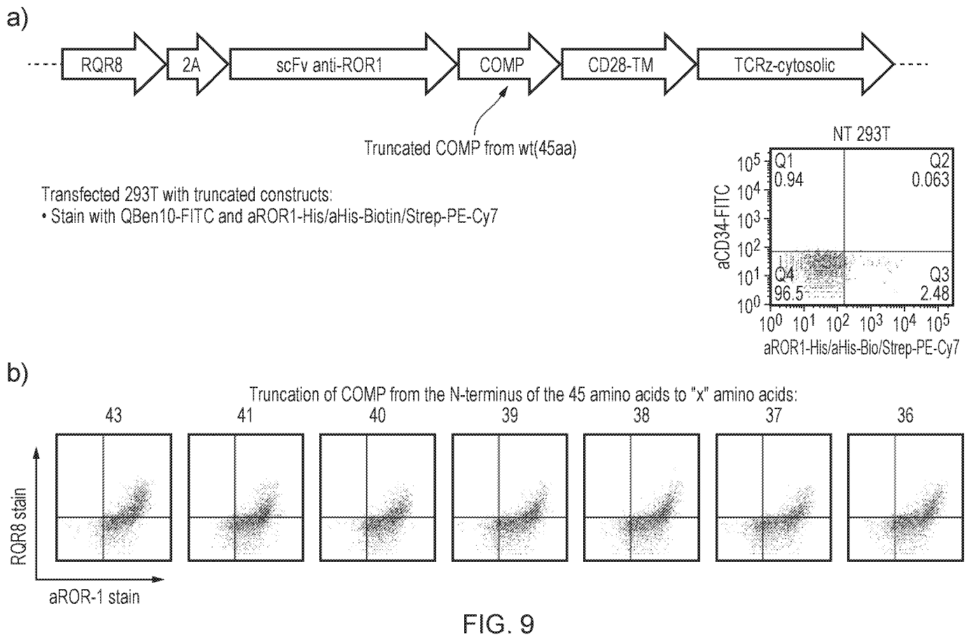

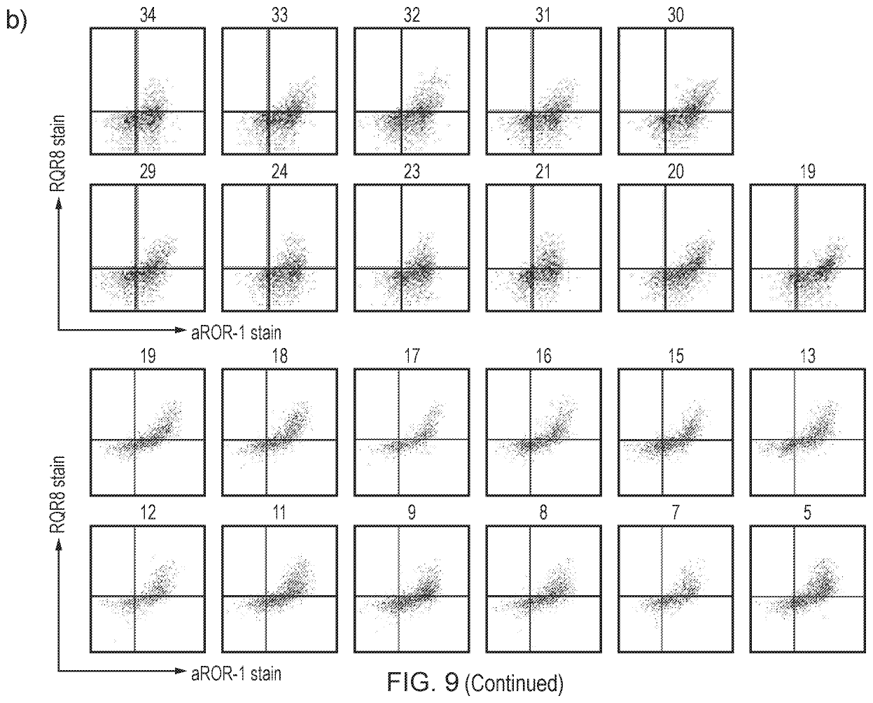

COMP is one of the most stable protein complexes in nature (stable from 0° C.-100° C. and a wide range of pH) and can only be denatured with 4-6M guanidine hydrochloride. The COMP coiled coil domain is capable of forming a pentamer. COMP is also an endogenously expressed protein that is naturally expressed in the extracellular space. This reduces the risk of immunogenicity compared to synthetic spacers. Furthermore, the crystal structure of the COMP coiled coil motif has been solved which gives an accurate estimation on the spacer length (FIG. 8). The COMP structure is ˜5.6 nm in length (compared to the hinge and CH2CH3 domains from human IgG which is ˜8.1 nm).

The coiled coil domain may consist of or comprise the sequence shown as SEQ ID No. 83 or a fragment thereof.

SEQ ID No. 83
DLGPQMLRELQETNAALQDVRELLRQQVREITFLKNTVMECDACG

As shown in FIG. 8, it is possible to truncate the COMP coiled-coil domain at the N-terminus and retain surface expression. The coiled-coil domain may therefore comprise or consist of a truncated version of SEQ ID No. 83, which is truncated at the N-terminus. The truncated COMP may comprise the 5 C-terminal amino acids of SEQ ID No. 83, i.e. the sequence CDACG. The truncated COMP may comprise 5 to 44 amino acids, for example, at least 5, 10, 15, 20, 25, 30, 35 or 40 amino acids. The truncated COMP may correspond to the C-terminus of SEQ ID No. 83. For example a truncated COMP comprising 20 amino acids may comprise the sequences QQVREITFLKNTVMECDACG (SEQ ID No. 84). Truncated COMP may retain the cysteine residue(s) involved in multimerisation. Truncated COMP may retain the capacity to form multimers.

Various coiled coil domains are known which form hexamers such as gp41derived from HIV, and an artificial protein designed hexamer coiled coil described by N. Zaccai et al. (2011) Nature Chem. Bio., (7) 935-941). A mutant form of the GCN4-p1 leucine zipper forms a heptameric coiled-coil structure (J. Liu. et al., (2006) PNAS (103) 15457-15462).

The coiled coil domain may comprise a variant of one of the coiled coil domains described above, providing that the variant sequence retains the capacity to form a coiled coil oligomer. For example, the coiled coil domain may comprise a variant of the sequence shown as SEQ ID No. 83 or 70 to 82 having at least 80, 85, 90, 95, 98 or 99% sequence identity, providing that the variant sequence retains the capacity to form a coiled coil oligomer.

The percentage identity between two polypeptide sequences may be readily determined by programs such as BLAST which is freely available at http://blast.ncbi.nlm.nih.gov.

CARs comprising coiled coil domains are described in more detail in WO2016/151315, the content of which is hereby incorporated by reference in its entirety.

### Transmembrane Domain

The transmembrane domain is the sequence of the CAR that spans the membrane.

A transmembrane domain may be any protein structure which is thermodynamically stable in a membrane. This is typically an alpha helix comprising of several hydrophobic residues. The transmembrane domain of any transmembrane protein can be used to supply the transmembrane portion of the invention. The presence and span of a transmembrane domain of a protein can be determined by those skilled in the art using the TMHMM algorithm (http://www.cbs.dtu.dk/services/TMHMM-2.0/). Further, given that the transmembrane domain of a protein is a relatively simple structure, i.e a polypeptide sequence predicted to form a hydrophobic alpha helix of sufficient length to span the membrane, an artificially designed TM domain may also be used (U.S. Pat. No. 7,052,906 B1 describes synthetic transmembrane components).

The transmembrane domain may be derived from CD28, which gives good receptor stability.

The transmembrane domain may be derived from human Tyrp-1. The tyrp-1 transmembrane sequence is shown as SEQ ID No. 85.

SEQ ID No. 85
IIAIAVVGALLLVALIFGTASYLI

### Activating Endodomain

The endodomain is the signal-transmission portion of the CAR. After antigen recognition, receptors cluster, native CD45 and CD148 are excluded from the synapse and a signal is transmitted to the cell. The most commonly used endodomain component is that of CD3-zeta which contains 3 ITAMs. This transmits an activation signal to the T cell after antigen is bound. CD3-zeta may not provide a fully competent activation signal and additional co-stimulatory signaling may be needed. For example, chimeric CD28 and OX40 can be used with CD3-Zeta to transmit a proliferative/survival signal, or all three can be used together.

The cell of the present invention comprises two CARs, each with an endodomain.

The endodomain of the first CAR may comprise:
(i) an ITAM-containing endodomain, such as the endodomain from CD3 zeta; and/or
(ii) a domain which transmits a survival signal, for example a TNF receptor family endodomain such as OX-40 or 4-1BB.

The endodomain of the second CAR may comprise:
(i) an ITAM-containing endodomain, such as the endodomain from CD3 zeta; and/or
(ii) a co-stimulatory domain, such as the endodomain from CD28.

In this arrangement the co-stimulatory and survival signal-producing domains are “shared” between the two (or more) CARs in an OR gate. For example, where an OR gate has two CARs, CAR A and CAR B, CAR A may comprise a co-stimulatory domain (e.g. CD28 endodomain) and CAR B may comprise a TNF receptor family endodomain, such as OX-40 or 4-1BB.

An endodomain which contains an ITAM motif can act as an activation endodomain in this invention. Several proteins are known to contain endodomains with one or more ITAM motifs. Examples of such proteins include the CD3 epsilon chain, the CD3 gamma chain and the CD3 delta chain to name a few. The ITAM motif can be easily recognized as a tyrosine separated from a leucine or isoleucine by any two other amino acids, giving the signature YxxL/I. Typically, but not always, two of these motifs are separated by between 6 and 8 amino acids in the tail of the molecule (YxxL/Ix(6-8)YxxL/I). Hence, one skilled in the art can readily find existing proteins which contain one or more ITAM to transmit an activation signal. Further, given the motif is simple and a complex secondary structure is not required, one skilled in the art can design polypeptides containing artificial ITAMs to transmit an activation signal (see WO 2000/063372, which relates to synthetic signalling molecules).

The transmembrane and intracellular T-cell signalling domain (endodomain) of a CAR with an activating endodomain may comprise the sequence shown as SEQ ID No. 86, 87 or 88 or a variant thereof having at least 80% sequence identity.

comprising CD28 transmembrane domain
and CD3 Z endodomain
SEQ ID No. 86
FWVLVVVGGVLACYSLLVTVAFIIFWVRRVKFSRSADAPAYQQGQ
NQLYNELNLGRREEYDVLDKRRGRDPEMGGKPRRKNPQEGLYNEL
QKDKMAEAYSEIGMKGERRRGKGHDGLYQGLSTATKDTYDALHMQ
ALPPR
comprising CD28 transmembrane domain
and CD28 and CD3 Zeta endodomains
SEQ ID No. 87
FWVLVVVGGVLACYSLLVTVAFIIFWVRSKRSRLLHSDYMNMTPR
RPGPTRKHYQPYAPPRDFAAYRSRVKFSRSADAPAYQQGQNQLYN
ELNLGRREEYDVLDKRRGRDPEMGGKPRRKNPQEGLYNELQKDKM
AEAYSEIGMKGERRRGKGHDGLYQGLSTATKDTYDALHMQALPPR
comprising CD28 transmembrane domain
and CD28, OX40 and CD3 Zeta endodomains.
SEQ ID No. 88
FWVLVVVGGVLACYSLLVTVAFIIFWVRSKRSRLLHSDYMNMTPR
RPGPTRKHYQPYAPPRDFAAYRSRDQRLPPDAHKPPGGGSFRTPI
QEEQADAHSTLAKIRVKFSRSADAPAYQQGQNQLYNELNLGRREE
YDVLDKRRGRDPEMGGKPRRKNPQEGLYNELQKDKMAEAYSEIGM
KGERRRGKGHDGLYQGLSTATKDTYDALHMQALPPR

A variant sequence may have at least 80%, 85%, 90%, 95%, 98% or 99% sequence identity to SEQ ID No. 86, 87 or 88, provided that the sequence provides an effective trans-membrane domain and an effective intracellular T cell signaling domain.

### “Split” or Gate Endodomains

The present invention provides an OR gate in which the co-stimulatory/survival signal domains are “split” between the two CARs.

In this respect, the present invention provides a cell which co-expresses at the cell surface a first chimeric antigen receptor (CAR) comprising an antigen-binding domain which binds to CD19, and a second CAR comprising an antigen-binding domain which binds to CD22, each CAR comprising an intracellular signalling domain, wherein the intracellular signalling domain of the first CAR comprises a TNF receptor family endodomain; and the intracellular signalling domain of the second CAR comprises a co-stimulatory domain.

The intracellular signalling domain of the first CAR comprises a TNF receptor family endodomain and does not comprise a co-stimulatory domain (such as CD28 endodomain). The intracellular signalling domain of the second CAR comprises a co-stimulatory domain and does not comprise a domain which transmits survival signals (such as a TNF receptor family endodomain).

The co-stimulatory domain may be a CD28 co-stimulatory domain. The CD28 co-stimulatory domain may have the sequence shown as SEQ ID No. 89.

(CD28 co-stimulatory endodomain)
SEQ ID No. 89
SKRSRLLHSDYMNMTPRRPGPTRKHYQPYAPPRDFAAYRS

The CAR of the invention may comprise a variant of the sequence shown as SEQ ID No. 89 having at least 80, 85, 90, 95, 98 or 99% sequence identity, provided that the variant sequence retains the capacity to co-stimulate T cells upon antigen recognition, i.e. provide signal 2 to T cells.

The TNF receptor family endodomain may be an OX40 or 4-1BB endodomain. The OX40 endodomain may have the sequence shown as SEQ ID No. 90. The 4-1BB endodomain may have the sequence shown as SEQ ID No. 91.

(OX40 endodomain)
SEQ ID No. 90
RDQRLPPDAHKPPGGGSFRTPIQEEQADAHSTLAKI
(4-1BB endodomain)
SEQ ID No. 91
KRGRKKLLYIFKQPFMRPVQTTQEEDGCSCRFPEEEEGGCEL

The CAR of the invention may comprise a variant of the sequence shown as SEQ ID No. 90 or 91 having at least 80, 85, 90, 95, 98 or 99% sequence identity, provided that the variant sequence retains the capacity to transmit a survival signal to T cells upon antigen recognition.

The intracellular signalling domain of the first and/or the second CAR may also comprise an ITAM-containing domain, such as a CD3 zeta domain. The CD3 zeta domain may have the sequence shown as SEQ ID No. 92.

(CD3zeta endodomain)
SEQ ID No. 92
RVKFSRSADAPAYQQGQNQLYNELNLGRREEYDVLDKRRGRDPEM
GGKPRRKNPQEGLYNELQKDKMAEAYSEIGMKGERRRGKGHDGLY
QGLSTATKDTYDALHMQALPPR

The CAR of the invention may comprise a variant of the sequence shown as SEQ ID No. 92 having at least 80, 85, 90, 95, 98 or 99% sequence identity, provided that the variant sequence retains the capacity to induce T-cell signalling upon antigen recognition, i.e. provide signal 1 to T cells.

The first CAR may have the structure:

AgB1-spacer1-TM1-TNF-ITAM
in which:
AgB1 is the antigen-binding domain of the first CAR;
spacer 1 is the spacer of the first CAR;
TM1 is the transmembrane domain of the first CAR;
TNF is a TNF receptor endodomain; and
ITAM is an ITAM-containing endodomain.

“TNF” may be a TNF receptor endodomain such as the OX40 or 4-1BB endodomains. “ITAM” may be a CD3 zeta endodomain.

The second CAR may have the structure:

AgB2-spacer2-TM2-costim-ITAM
in which:
AgB2 is the antigen-binding domain of the second CAR;
spacer 2 is the spacer of the second CAR;
TM2 is the transmembrane domain of the second CAR;
costim is a co-stimulatory domain; and
ITAM is an ITAM-containing endodomain.

“Costim” may be a CD28 co-stimulatory domain.

There is also provided a nucleic acid sequence encoding both the first and second chimeric antigen receptors (CARs) with “split” endodomains; and a kit comprising two nucleic acids one encoding a first CAR and one encoding a second CAR comprising split endodomains as defined above.

### Co-Expression Site

The second aspect of the invention relates to a nucleic acid which encodes the first and second CARs.

The nucleic acid may produce a polypeptide which comprises the two CAR molecules joined by a cleavage site. The cleavage site may be self-cleaving, such that when the polypeptide is produced, it is immediately cleaved into the first and second CARs without the need for any external cleavage activity.

Various self-cleaving sites are known, including the Foot-and-Mouth disease virus (FMDV) 2A peptide and similar sequences (Donnelly et al, Journal of General Virology (2001), 82, 1027-1041), for instance like the 2A-like sequence from Thosea asigna virus which has the sequence shown as SEQ ID No. 12:

SEQ ID No. 93
RAEGRGSLLTCGDVEENPGP.

These sequences may also be referred to as cis-acting hydrolase element (CHYSEL) sequences.

The co-expressing sequence may be an internal ribosome entry sequence (IRES). The co-expressing sequence may be an internal promoter.

Nucleic acid constructs may contain multiple co-expression sites leading to the production of multiple polypeptides. For example, a construct may include multiple 2A-like sequences, which may be the same or different.

### Modulating the Activity of the CAR

### Enhancing ITAM Phosphorylation

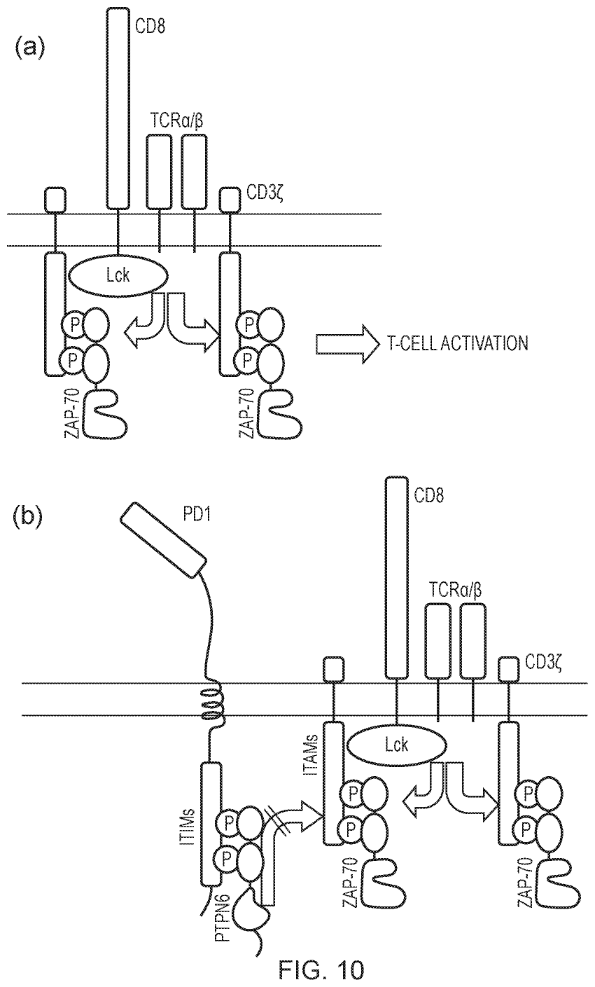

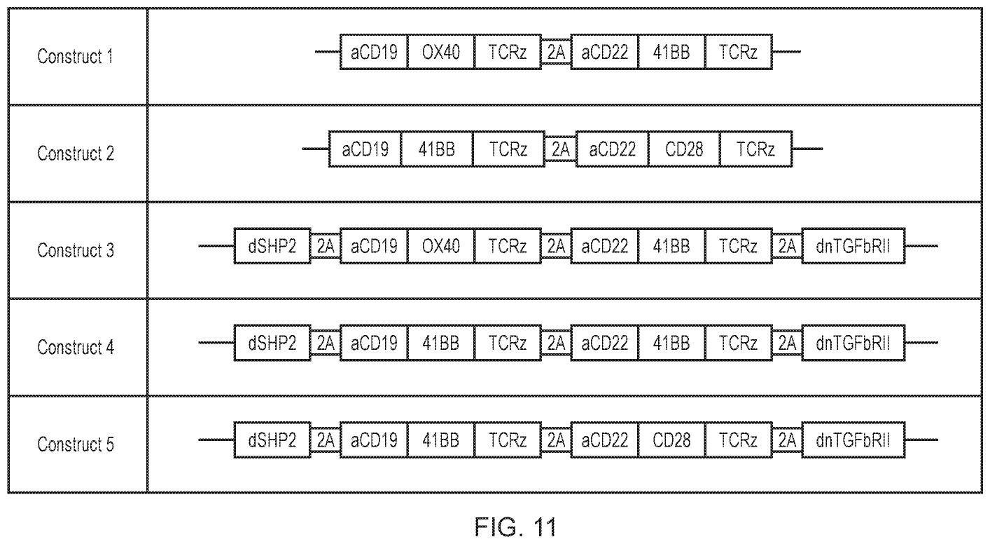

During T cell activation in vivo (illustrated schematically in FIG. 10a), antigen recognition by the T-cell receptor (TCR) results in phosphorylation of Immunoreceptor tyrosine-based activation motifs (ITAMs) on CD3ζ. Phosphorylated ITAMs are recognized by the ZAP70 SH2 domains, leading to T cell activation.

T-cell activation uses kinetic segregation to convert antigen recognition by a TCR into downstream activation signals. Briefly: at the ground state, the signalling components on the T-cell membrane are in dynamic homeostasis whereby dephosphorylated ITAMs are favoured over phosphorylated ITAMs. This is due to greater activity of the transmembrane CD45/CD148 phosphatases over membrane-tethered kinases such as Ick. When a T-cell engages a target cell through a T-cell receptor (or CAR) recognition of cognate antigen, tight immunological synapses form. This close juxtapositioning of the T-cell and target membranes excludes CD45/CD148 due to their large ectodomains which cannot fit into the synapse. Segregation of a high concentration of T-cell receptor associated ITAMs and kinases in the synapse, in the absence of phosphatases, leads to a state whereby phosphorylated ITAMs are favoured. ZAP70 recognizes a threshold of phosphorylated ITAMs and propagates a T-cell activation signal.

The process is essentially the same during CAR-mediated T-cell activation. An activating CAR comprises one or more ITAM(s) in its intracellular signalling domain, usually because the signalling domain comprises the endodomain of CD3ζ. Antigen recognition by the CAR results in phosphorylation of the ITAM(s) in the CAR signalling domain, causing T-cell activation.

As illustrated schematically in FIG. 10b, inhibitory immune-receptors such as PD1 cause the dephosphorylation of phosphorylated ITAMs. PD1 has ITIMs in its endodomain which are recognized by the SH2 domains of molecules such as PTPN6 (SHP-1) and PTPN11 (SHP-2). Upon recognition, PTPN6 is recruited to the juxta-membrane region and its phosphatase domain subsequently de-phosphorylates ITAM domains inhibiting immune activation.

### Modulating the Activity of the CAR-T Cell

### Checkpoint Inhibition

CAR-mediated T-cell activation is mediated by inhibitory immunoreceptors such as CTLA4, PD-1, LAG-3, 2B4 or BTLA 1 (as mentioned above and illustrated schematically in FIG. 10b).

### PD-1/PD-L1

In the cancer disease state, the interaction of PD-L1 on the tumour cells with PD-1 on a T-cell reduces T-cell activation, as described above, thus hampering the immune system in its efforts to attack the tumour cells. Use of an inhibitor that blocks the interaction of PD-L1 with the PD-1 receptor can prevent the cancer from evading the immune system in this way. Several PD-1 and PD-L1 inhibitors are being trialled within the clinic for use in advanced melanoma, non-small cell lung cancer, renal cell carcinoma, bladder cancer and Hodgkin lymphoma, amongst other cancer types. Some such inhibitors are now approved, including the PD1 inhibitors Nivolumab and Pembrolizumab and the PD-L1 inhibitors Atezolizumab, Avelumab and Durvalumab.

### CTLA4

CTLA4 is a member of the immunoglobulin superfamily that is expressed by activated T cells and transmits an inhibitory signal to T cells. CTLA4 is homologous to the T-cell co-stimulatory protein, CD28, and both molecules bind to CD80 and CD86, also called B7-1 and B7-2 respectively, on antigen-presenting cells. CTLA-4 binds CD80 and CD86 with greater affinity and avidity than CD28 thus enabling it to outcompete CD28 for its ligands. CTLA4 transmits an inhibitory signal to T cells, whereas CD28 transmits a stimulatory signal.

Antagonistic antibodies against CTLA4 include ipilimumab and tremelimumab.

### LAG-3

Lymphocyte-activation gene 3, also known as LAG-3 and CD223, is an immune checkpoint receptor with diverse biologic effects on T-cell function.

Antibodies to LAG3 include relatlimab, which currently in phase 1 clinical testing and a number of others in preclinical development. LAG-3 may be a better checkpoint inhibitor target than CTLA-4 or PD-1 since antibodies to these two checkpoints only activate effector T cells, and do not inhibit Treg activity, whereas an antagonist LAG-3 antibody can both activate T effector cells (by downregulating the LAG-3 inhibiting signal into pre-activated LAG-3+ cells) and inhibit induced (i.e. antigen-specific) Treg suppressive activity. Combination therapies are also ongoing involving LAG-3 antibodies and CTLA-4 or PD-1 antibodies.

### Dominant Negative SHP

WO2016/193696 describes various different types of protein capable of modulating the balance of phosphorylation:dephosporylation at the T-cell:target cell synapse. For example, WO2016/193696 describes truncated forms of SHP-1 or SHP-2 which comprises one or both SH2 domains, but lacks the phosphatase domain. When expressed in a CAR-T cell, these molecules act as dominant negative versions of wild-type SHP-1 and SHP-2 and compete with the endogenous molecule for binding to phosphorylated ITIMs.

These dominant negative versions of wild-type SHP-1 and SHP-2 block or reduce the inhibition mediated by inhibitory immunoreceptors such as CTLA4, PD-1, LAG-3, 2B4 or BTLA 1 and tip the balance of phosphorylation:dephosporylation at the T-cell:target cell synapse in favour of phosphorylation of ITAMs, leading to T-cell activation.

The cell of the present invention may express a truncated protein which comprises an SH2 domain from a protein which binds a phosphorylated immunoreceptor tyrosine-based inhibition motif (ITIM) but lacks a phosphatase domain. The truncated protein may comprise one or both SHP-1 SH2 domain(s) but lack the SHP-1 phosphatase domain. Alternatively the truncated protein may comprise one or both SHP-2 SH2 domain(s) but lack the SHP-2 phosphatase domain.

### SHP-1

Src homology region 2 domain-containing phosphatase-1 (SHP-1) is a member of the protein tyrosine phosphatase family. It is also known as PTPN6.

The N-terminal region of SHP-1 contains two tandem SH2 domains which mediate the interaction of SHP-1 and its substrates. The C-terminal region contains a tyrosine-protein phosphatase domain.

SHP-1 is capable of binding to, and propagating signals from, a number of inhibitory immune receptors or ITIM containing receptors. Examples of such receptors include, but are not limited to, PD1, PDCD1, BTLA4, LILRB1, LAIR1, CTLA4, KIR2DL1, KIR2DL4, KIR2DL5, KIR3DL1 and KIR3DL3.

Human SHP-1 protein has the UniProtKB accession number P29350.

Truncated SHP-1 may comprise or consist of the SHP-1 tandem SH2 domain which is shown below as SEQ ID NO: 94.

SHP-1 SH2 complete domain
(SEQ ID NO: 94)
MVRWFHRDLSGLDAETLLKGRGVHGSFLARPSRKNQGDFSLSVRV
GDQVTHIRIQNSGDFYDLYGGEKFATLTELVEYYTQQQGVLQDRD
GTIIHLKYPLNCSDPTSERWYHGHMSGGQAETLLQAKGEPWTFLV
RESLSQPGDFVLSVLSDQPKAGPGSPLRVTHIKVMCEGGRYTVGG
LETFDSLTDLVEHFKKTGIEEASGAFVYLRQPYY

SHP-1 has two SH2 domains at the N-terminal end of the sequence, at residues 4-100 and 110-213. Truncated SHP-1 may comprise one or both of the sequences shown as SEQ ID No. 95 and 96.

SHP-1 SH2 1
(SEQ ID NO: 95)
WFHRDLSGLDAETLLKGRGVHGSFLARPSRKNQGDFSLSVRVGDQ
VTHIRIQNSGDFYDLYGGEKFATLTELVEYYTQQQGVLQDRDGTI
IHLKYPL
SHP-1 SH2 2
(SEQ ID No. 96)
WYHGHMSGGQAETLLQAKGEPWTFLVRESLSQPGDFVLSVLSDQP
KAGPGSPLRVTHIKVMCEGGRYTVGGLETFDSLTDLVEHFKKTGI
EEASGAFVYLRQPY

The truncated SHP-1 may comprise a variant of SEQ ID NO: 94, 95 or 96 having at least 80, 85, 90, 95, 98 or 99% sequence identity, provided that the variant sequence is a SH2 domain sequence has the required properties. In other words, the variant sequence should be capable of binding to the phosphorylated tyrosine residues in the cytoplasmic tail of at least one of PD1, PDCD1, BTLA4, LILRB1, LAIR1, CTLA4, KIR2DL1, KIR2DL4, KIR2DL5, KIR3DL1 or KIR3DL3 which allow the recruitment of SHP-1.

### SHP-2

SHP-2, also known as PTPN11, PTP-1D and PTP-2C is a member of the protein tyrosine phosphatase (PTP) family. Like PTPN6, SHP-2 has a domain structure that consists of two tandem SH2 domains in its N-terminus followed by a protein tyrosine phosphatase (PTP) domain. In the inactive state, the N-terminal SH2 domain binds the PTP domain and blocks access of potential substrates to the active site. Thus, SHP-2 is auto-inhibited. Upon binding to target phospho-tyrosyl residues, the N-terminal SH2 domain is released from the PTP domain, catalytically activating the enzyme by relieving the auto-inhibition.

Human SHP-2 has the UniProtKB accession number P35235-1.

Truncated SHP-2 may comprise or consist of the SHP-1 tandem SH2 domain which is shown below as SEQ ID NO: 99. SHP-1 has two SH2 domains at the N-terminal end of the sequence, at residues 6-102 and 112-216. Truncated SHP-2 may comprise one or both of the sequences shown as SEQ ID No. 97 and 98.

SHP-2 first SH2 domain
(SEQ ID NO: 97)
WFHPNITGVEAENLLLTRGVDGSFLARPSKSNPGDFTLSVRRNGA
VTHIKIQNTGDYYDLYGGEKFATLAELVQYYMEHHGQLKEKNGDV
IELKYPL
SHP-2 second SH2 domain
(SEQ ID No. 98)
WFHGHLSGKEAEKLLTEKGKHGSFLVRESQSHPGDFVLSVRTGDD
KGESNDGKSKVTHVMIRCQELKYDVGGGERFDSLTDLVEHYKKNP
MVETLGTVLQLKQPL
SHP-2 both SH2 domains
(SEQ ID No. 99)
WFHPNITGVEAENLLLTRGVDGSFLARPSKSNPGDFTLSVRRNGA
VTHIKIQNTGDYYDLYGGEKFATLAELVQYYMEHHGQLKEKNGDV
IELKYPLNCADPTSERWFHGHLSGKEAEKLLTEKGKHGSFLVRES
QSHPGDFVLSVRTGDDKGESNDGKSKVTHVMIRCQELKYDVGGGE
RFDSLTDLVEHYKKNPMVETLGTVLQLKQPL

Truncated SHP-2 may comprise a variant of SEQ ID NO: 97, 98 or 99 having at least 80, 85, 90, 95, 98 or 99% sequence identity, provided that the variant sequence is a SH2 domain sequence has the required properties. In other words, the variant sequence should be capable of binding to the phosphorylated tyrosine residues in the cytoplasmic tail of at least one of PD1, PDCD1, BTLA4, LILRB1, LAIR1, CTLA4, KIR2DL1, KIR2DL4, KIR2DL5, KIR3DL1 or KIR3DL3 which allow the recruitment of SHP-2.

### Modulating TGFβ Signalling

Engineered cells face hostile microenvironments which limit adoptive immunotherapy. One of the main inhibitory mechanisms within the tumour microenvironment is transforming growth factor beta (TGFβ). The TGFβ signalling pathway has a pivotal role in the regulatory signalling that controls a variety of cellular processes. TGFβ play also a central role in T cell homeostasis and control of cellular function. Particularly, TGFβ signalling is linked to an immuno-depressed state of the T-cells, with reduced proliferation and activation. TGFβ expression is associated with the immunosuppressive microenvironment of tumour.

A variety of cancerous tumour cells are known to produce TGFβ directly. In addition to the TGFβ production by cancerous cells, TGFβ can be produced by the wide variety of non-cancerous cells present at the tumour site such as tumour-associated T cells, natural killer (NK) cells, macrophages, epithelial cells and stromal cells.

The transforming growth factor beta receptors are a superfamily of serine/threonine kinase receptors. These receptors bind members of the TGFβ superfamily of growth factor and cytokine signalling proteins. There are five type II receptors (which are activatory receptors) and seven type I receptors (which are signalling propagating receptors).

Auxiliary co-receptors (also known as type III receptors) also exist. Each subfamily of the TGFβ superfamily of ligands binds to type I and type II receptors.

The three transforming growth factors have many activities. TGFβ1 and 2 are implicated in cancer, where they may stimulate the cancer stem cell, increase fibrosis/desmoplastic reactions and suppress immune recognition of the tumour.

TGFβ1, 2 and 3 signal via binding to receptors TβRII and then association to TβRI and in the case of TGFβ2 also to TβRIII. This leads to subsequent signalling through SMADs via TβRI.

TGFβs are typically secreted in the pre-pro-form. The “pre” is the N-terminal signal peptide which is cleaved off upon entry into the endoplasmic reticulum (ER). The “pro” is cleaved in the ER but remains covalently linked and forms a cage around the TGFβ called the Latency Associated Peptide (LAP). The cage opens in response to various proteases including thrombin and metalloproteases amongst others. The C-terminal region becomes the mature TGFβ molecule following its release from the pro-region by proteolytic cleavage. The mature TGFβ protein dimerizes to produce an active homodimer.

The TGFβ homodimer interacts with a LAP derived form the N-terminal region of the TGFβ gene product, forming a complex called Small Latent Complex (SLC). This complex remains in the cell until it is bound by another protein, an extracellular matrix (ECM) protein called Latent TGFβ binding protein (LTBP) which together forms a complex called the large latent complex (LLC). LLC is secreted to the ECM. TGFβ is released from this complex to a biologically active form by several classes of proteases including metalloproteases and thrombin.

### Dominant Negative TGFβ Receptor

The active TGFβ receptor (TβR) is a hetero-tetramer, composed by two TGFβ receptor I (TβRI) and two TGFβ receptor II (TβRII). TGFβ1 is secreted in a latent form and is activated by multiple mechanisms. Once activated it forms a complex with the TβRII TβRI that phosphorylates and activates TβRI.

The cell of the present invention expresses dominant negative TGFβ receptor. A dominant negative TGFβ receptor may lack the kinase domain.

For example, the dominant negative TGFβ receptor may comprise or consist of the sequence shown as SEQ ID No. 100, which is a monomeric version of TGF receptor II

(dn TGFβ RII)
SEQ ID No. 100
TIPPHVQKSVNNDMIVTDNNGAVKFPQLCKFCDVRFSTCDNQKSC
MSNCSITSICEKPQEVCVAVWRKNDENITLETVCHDPKLPYHDFI
LEDAASPKCIMKEKKKPGETFFMCSCSSDECNDNIIFSEEYNTSN
PDLLLVIFQVTGISLLPPLGVAISVIIIFYCYRVNRQQKLSS

A dominant-negative TGF-βRII (dnTGF-βRII) has been reported to enhance PSMA targeted CAR-T cell proliferation, cytokine secretion, resistance to exhaustion, long-term in vivo persistence, and the induction of tumour eradication in aggressive human prostate cancer mouse models (Kloss et al (2018) Mol. Ther.26:1855-1866).

### Cell

The present invention relates to a cell which co-expresses a first CAR and a second CAR at the cell surface, wherein one CAR binds CD19 and the other CAR binds CD22.

The cell may be any eukaryotic cell capable of expressing a CAR at the cell surface, such as an immunological cell.

In particular the cell may be an immune effector cell such as a T cell or a natural killer (NK) cell.

T cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytotoxic T cells (TC cells, or CTLs) destroy virally infected cells and tumor cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells.

Naturally occurring Treg cells (also known as CD4+CD25+FoxP3+ Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Tr1 cells or Th3 cells) may originate during a normal immune response.

Natural killer T (NKT) cells are a heterogeneous group of T cells that share properties of both T cells and natural killer cells. Many of these cells recognize the non-polymorphic CD1d molecule, an antigen-presenting molecule that binds self and foreign lipids and glycolipids.

The T cell of the invention may be any of the T cell types mentioned above, in particular a CTL.

Natural killer (NK) cells are a type of cytolytic cell which forms part of the innate immune system. NK cells provide rapid responses to innate signals from virally infected cells in an MHC independent manner

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The CAR cells of the invention may be any of the cell types mentioned above.

CAR-expressing cells, such as CAR-expressing T or NK cells may either be created ex vivo either from a patient's own peripheral blood (1st party), or in the setting of a haematopoietic stem cell transplant from donor peripheral blood (2nd party), or peripheral blood from an unconnected donor (3rd party).

The present invention also provide a cell composition comprising CAR expressing T cells and/or CAR expressing NK cells according to the present invention. The cell composition may be made by transducing a blood-sample ex vivo with a nucleic acid according to the present invention.

Alternatively, CAR-expressing cells may be derived from ex vivo differentiation of inducible progenitor cells or embryonic progenitor cells to the relevant cell type, such as T cells. Alternatively, an immortalized cell line such as a T-cell line which retains its lytic function and could act as a therapeutic may be used.

In all these embodiments, CAR cells are generated by introducing DNA or RNA coding for the CARs by one of many means including transduction with a viral vector, transfection with DNA or RNA.

A CAR T cell of the invention may be an ex vivo T cell from a subject. The T cell may be from a peripheral blood mononuclear cell (PBMC) sample. T cells may be activated and/or expanded prior to being transduced with CAR-encoding nucleic acid, for example by treatment with an anti-CD3 monoclonal antibody.

A CAR T cell of the invention may be made by:
(i) isolation of a T cell-containing sample from a subject or other sources listed above; and
(ii) transduction or transfection of the T cells with one or more nucleic acid sequence(s) encoding the first and second CAR.

The T cells may then by purified, for example, selected on the basis of co-expression of the first and second CAR.

### Nucleic Acid Sequences

The second aspect of the invention relates to one or more nucleic acid sequence(s) which codes for a first CAR and a second CAR as defined in the first aspect of the invention.

The nucleic acid sequence may be, for example, an RNA, a DNA or a cDNA sequence.

The nucleic acid sequence may encode one chimeric antigen receptor (CAR) which binds to CD19 and another CAR which binds to CD22.

The nucleic acid sequence may have the following structure:

AgB1-spacer1-TM1-coexpr-AbB2-spacer2-TM2
in which
AgB1 is a nucleic acid sequence encoding the antigen-binding domain of a first CAR;
spacer 1 is a nucleic acid sequence encoding the spacer of a first CAR;
TM1 is a a nucleic acid sequence encoding the transmembrane domain of a first CAR;
coexpr is a nucleic acid sequence enabling co-expression
AgB2 is a nucleic acid sequence encoding the antigen-binding domain of a second CAR;
spacer 2 is a nucleic acid sequence encoding the spacer of a second CAR;
TM2 is a a nucleic acid sequence encoding the transmembrane domain of a second CAR;
which nucleic acid sequence, when expressed in a T cell, encodes a polypeptide which is cleaved at the cleavage site such that the first and second CARs are co-expressed at the cell surface.

Alternatively, the nucleic acid sequence may have the following structure:

AbB2-spacer2-TM2-coexpr-AgB1-spacer1-TM1

In which the components AgB1, spacer1, TM1, coexpr, AbB2, spacer2, and TM2 are as defined above.

Alternative codons may be used in regions of sequence encoding the same or similar amino acid sequences, in order to avoid homologous recombination.

Due to the degeneracy of the genetic code, it is possible to use alternative codons which encode the same amino acid sequence. For example, the codons “ccg” and “cca” both encode the amino acid proline, so using “ccg” may be exchanged for “cca” without affecting the amino acid in this position in the sequence of the translated protein.

The alternative RNA codons which may be used to encode each amino acid are summarised in Table 4.

TABLE 4

| | U | C | A | G |
|---|---|---|---|---|
| U | 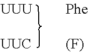 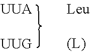 | 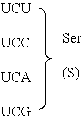 | 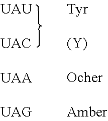 | 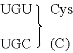 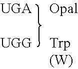 |
| C | 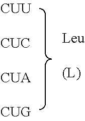 | 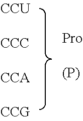 | 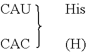 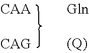 | 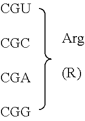 |
| A | 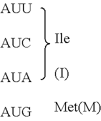 | 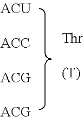 | 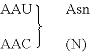 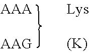 | 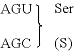 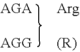 |
| G | 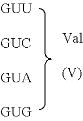 | 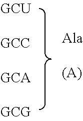 | 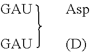 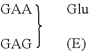 | 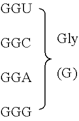 |

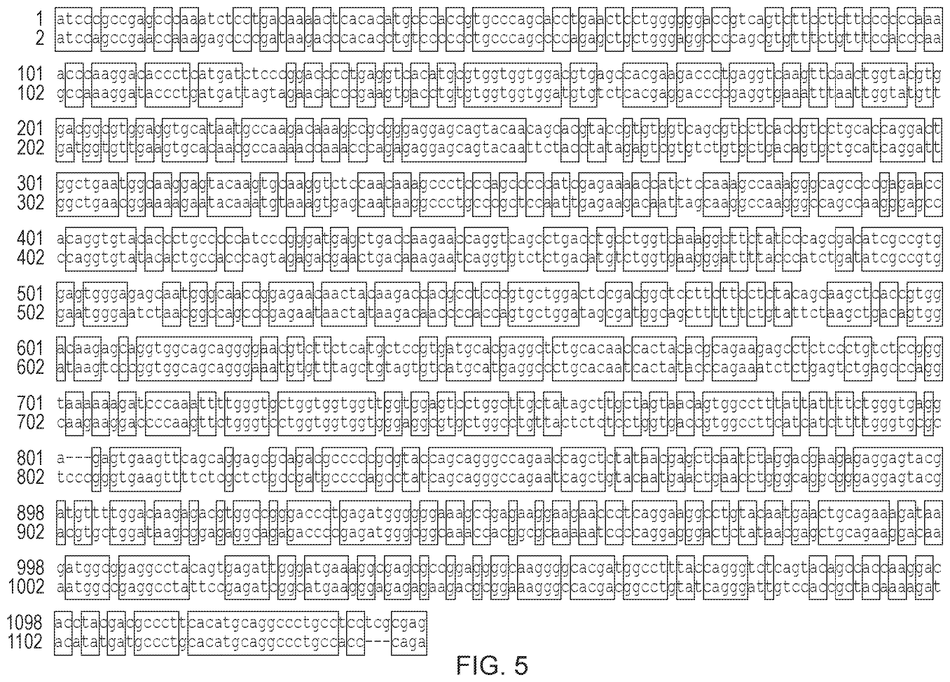

Alternative codons may be used in the portions of nucleic acid sequence which encode the spacer of the first CAR and the spacer of the second CAR, especially if the same or similar spacers are used in the first and second CARs. FIG. 5 shows two sequences encoding the spacer HCH2CH3— hinge, in one of which alternative codons have been used.

Alternative codons may be used in the portions of nucleic acid sequence which encode the transmembrane domain of the first CAR and the transmembrane of the second CAR, especially if the same or similar transmembrane domains are used in the first and second CARs. FIG. 5 shows two sequences encoding the CD28 transmembrane domain, in one of which alternative codons have been used.

Alternative codons may be used in the portions of nucleic acid sequence which encode all or part of the endodomain of the first CAR and all or part of the endodomain of the second CAR. Alternative codons may be used in the CD3 zeta endodomain. FIG. 5 shows two sequences encoding the CD3 zeta endodomain, in one of which alternative codons have been used.

Alternative codons may be used in one or more co-stimulatory domains, such as the CD28 endodomain.

Alternative codons may be used in one or more domains which transmit survival signals, such as OX40 and 41BB endodomains.

Alternative codons may be used in the portions of nucleic acid sequence encoding a CD3zeta endodomain and/or the portions of nucleic acid sequence encoding one or more costimulatory domain(s) and/or the portions of nucleic acid sequence encoding one or more domain(s) which transmit survival signals.

### Vector

The present invention also provides a vector, or kit of vectors which comprises one or more CAR-encoding nucleic acid sequence(s). Such a vector may be used to introduce the nucleic acid sequence(s) into a host cell so that it expresses the first and second CARs.

The vector may, for example, be a plasmid or a viral vector, such as a retroviral vector or a lentiviral vector, or a transposon based vector or synthetic mRNA.

The vector may be capable of transfecting or transducing a T cell.

### Pharmaceutical Composition

The present invention also relates to a pharmaceutical composition containing a plurality of CAR-expressing cells, such as T cells or NK cells according to the first aspect of the invention. The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

### Method of Treatment

The cells of the present invention are capable of killing cancer cells, such as B-cell lymphoma cells. CAR-expressing cells, such as T cells, may either be created ex vivo either from a patient's own peripheral blood (1st party), or in the setting of a haematopoietic stem cell transplant from donor peripheral blood (2nd party), or peripheral blood from an unconnected donor (3rd party). Alternatively, CAR T-cells may be derived from ex-vivo differentiation of inducible progenitor cells or embryonic progenitor cells to T-cells. In these instances, CAR T-cells are generated by introducing DNA or RNA coding for the CAR by one of many means including transduction with a viral vector, transfection with DNA or RNA.

The cells of the present invention may be capable of killing target cells, such as cancer cells. The target cell is recognisable by expression of CD19 or CD22.

TABLE 5
expression of lymphoid antigens on lymphoid leukaemias

| | CD19 | CD22 | CD10 | CD7 | CD5 | CD3 | clg μ | slg μ |
|---|---|---|---|---|---|---|---|---|
| Early pre-B | 100 | >95 | 95 | 5 | 0 | 0 | 0 | 0 |
| Pre-B | 100 | 100 | >95 | 0 | 0 | 0 | 100 | 0 |
| Transitional | 100 | 100 | 50 | 0 | 0 | 0 | 100 | 0 |
| pre-B | | | | | | | | |
| B | 100 | 100 | 50 | 0 | 0 | 0 | >95 | >95 |
| T | <5 | 0 | 0 | 100 | 95 | 100 | 0 | 0 |

Taken from Campana et al. (Immunophenotyping of leukemia. J. Immunol. Methods 243, 59-75 (2000)). clg μ-cytoplasic Immunoglobulin heavy chain; slg μ-surface Immunoglobulin heavy chain.

The expression of commonly studied lymphoid antigens on different types of B-cell leukaemias closely mirrors that of B-cell ontogeny (see FIG. 2).

The T cells of the present invention may be used to treat cancer, in particular B-cell malignancies.

Examples of cancers which express CD19 or CD22 are B-cell lymphomas, including Hodgkin's lymphoma and non-Hodgkins lymphoma; and B-cell leukaemias.

For example the B-cell lymphoma may be Diffuse large B cell lymphoma (DLBCL), Follicular lymphoma, Marginal zone lymphoma (MZL) or Mucosa-Associated Lymphatic Tissue lymphoma (MALT), Small cell lymphocytic lymphoma (overlaps with Chronic lymphocytic leukemia), Mantle cell lymphoma (MCL), Burkitt lymphoma, Primary mediastinal (thymic) large B-cell lymphoma, Lymphoplasmacytic lymphoma (may manifest as Waldenström macroglobulinemia), Nodal marginal zone B cell lymphoma (NMZL), Splenic marginal zone lymphoma (SMZL), Intravascular large B-cell lymphoma, Primary effusion lymphoma, Lymphomatoid granulomatosis, T cell/histiocyte-rich large B-cell lymphoma or Primary central nervous system lymphoma.

The B-cell leukaemia may be acute lymphoblastic leukaemia, B-cell chronic lymphocytic leukaemia, B-cell prolymphocytic leukaemia, precursor B lymphoblastic leukaemia or hairy cell leukaemia.

The B-cell leukaemia may be acute lymphoblastic leukaemia.

Treatment with the T cells of the invention may help prevent the escape or release of tumour cells which often occurs with standard approaches.

The invention will now be further described by way of Examples, which are meant to serve to assist one of ordinary skill in the art in carrying out the invention and are not intended in any way to limit the scope of the invention.

### EXAMPLES

### Example 1—Preparation of CD19/CD22 Logical ‘OR’ Gate Constructs and Target Cells

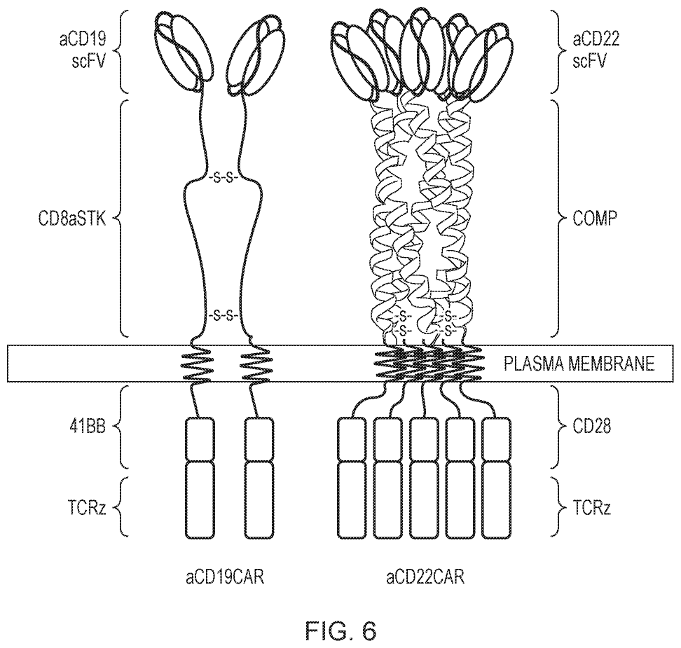

A CD19 ‘OR’ CD22 gate was constructed in which the CD19 CAR carries a TNFR family endodomain (4-1BB) and the CD22 CAR carries a co-stimulatory endodomain (CD28). The structure of each CAR is given in FIG. 6.

Several CD19/CD22 OR gate constructs were prepared as shown in FIG. 11 and summarised in Table 6. The first construct comprises a CD19 CAR and a CD22 CAR as described in WO2016/102965 (Construct 1, FIG. 11). The second construct comprises the CD19 CAR and CD22 CAR as shown in FIG. 6 (Construct 2, FIG. 11). Three further constructs were prepared, which additionally include a dominant negative SHP2 module (dSHP2) and a dominant negative TGFβRII module (dnTGFβRII) (Constructs 3, 4, and 5, FIG. 11). Co-expression was achieved by cloning the two CARs in frame separated by a 2A peptide

TABLE 6
Structure of CD19/CD22 CAR OR gate constructs

| ` | CD19 CAR | CD19 CAR | CD22 CAR | CD22 CAR |
|---|---|---|---|---|
| Construct | spacer | endodomain | spacer | endodomain |
| 1 | CD8 Stalk | OX40-CD3ζ | COMP | 41BB-CD3ζ |
| 2 | CD8 Stalk | 41BB-CD3ζ | COMP | CD28-CD3ζ |
| 3 | CD8 Stalk | OX40-CD3ζ | COMP | 41BB-CD3ζ |
| 4 | CD8 Stalk | 41BB-CD3ζ | COMP | 41BB-CD3ζ |
| 5 | CD8 Stalk | 41BB-CD3ζ | COMP | CD28-CD3ζ |

### Example 2—Comparison of CAR Endodomains

In order to identify optimal endodomains for a dual-targeting CD19/CD22 CAR-T cell, the ability of T cells expressing one of Constructs 1, 3, 4, or 5 to kill CD19+ or CD22+ SupT1 cells were compared. In addition, proliferation of T cells expressing one of Constructs 1, 3, 4, or 5 in the presence of CD19+ or CD22+ SupT1 cells was investigated.

Cells expressing the one of the constructs were co-cultured for 72 hours with target cells at a 1:1 effector:target (E:T) cell ratio (50,000 target cells).

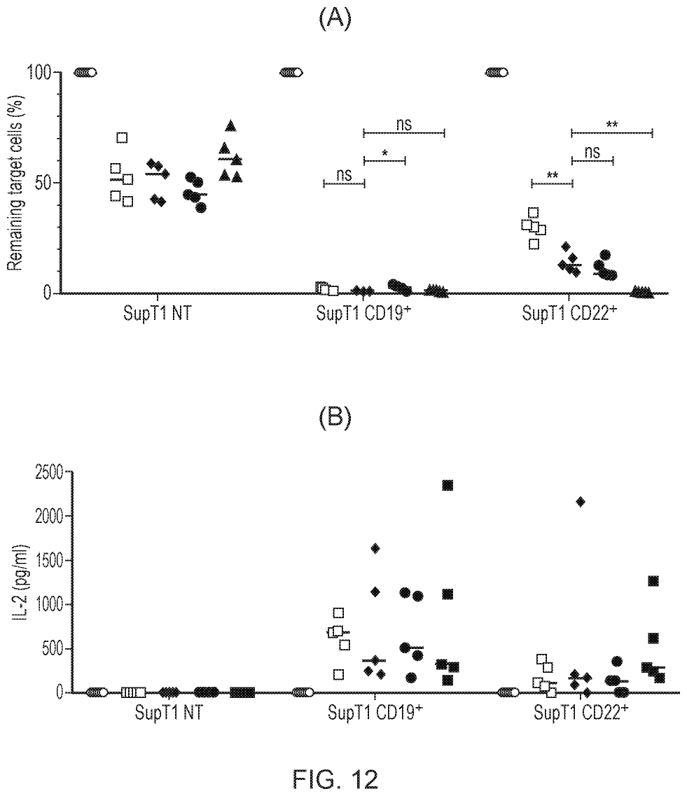

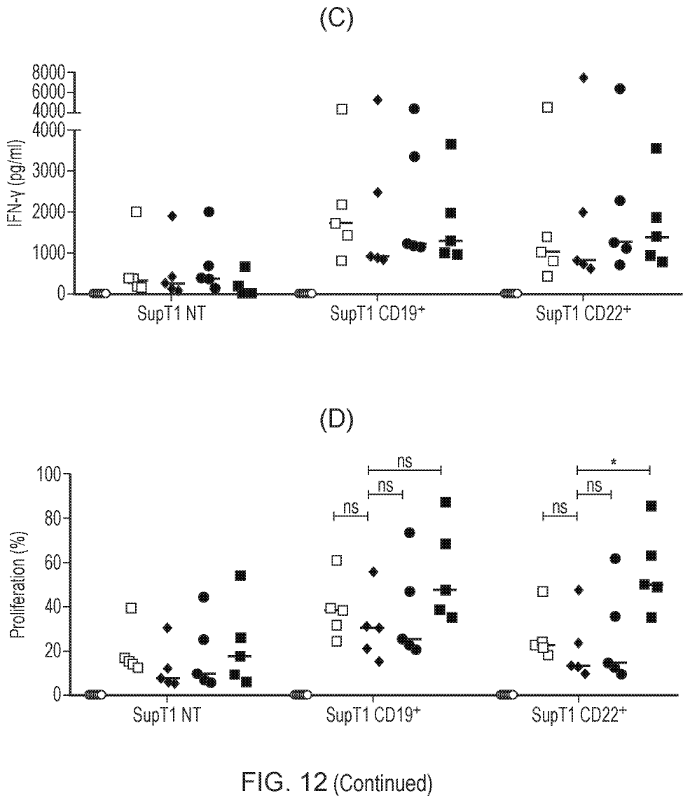

Results are shown in FIG. 12. While all constructs were able to kill CD19+ target cells, these results demonstrate that Construct 5 shows improved killing of CD22+ target cells compared to Constructs 1, 3, and 4. Proliferation of cells expressing Construct 5 was also improved. Levels of IL-2 and IFN-γ were similar for all constructs.

### Example 3—Further In Vitro Analysis

Cells expressing either Construct 1, 3, or 5 were tested against the following target cells in vitro:
Raji cells (CD19/CD22 positive cancer cell line);
CD19 knock-out Raji cells;
SupT1 high density CD19;
SupT1 low density CD19;
SupT1 high density CD22; and
SupT1 low density CD22.

Transduced PBMCs expressing the one of the constructs were co-cultured for 72 hours with target cells at both a 1:1 and 1:10 effector:target cell ratio.

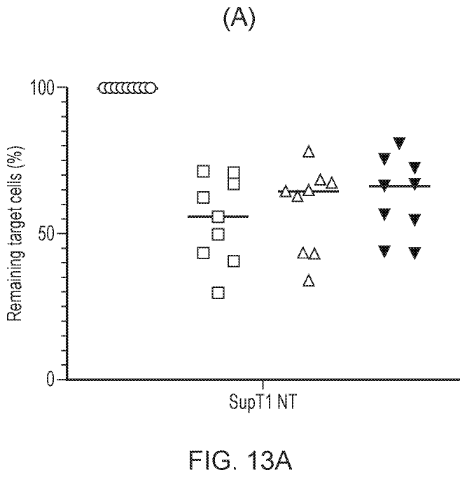

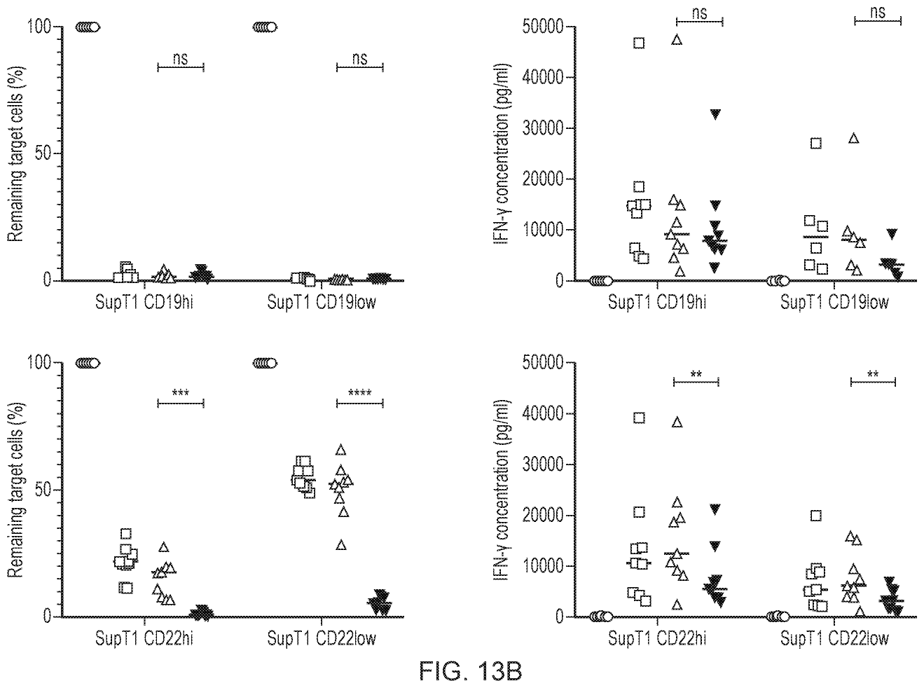

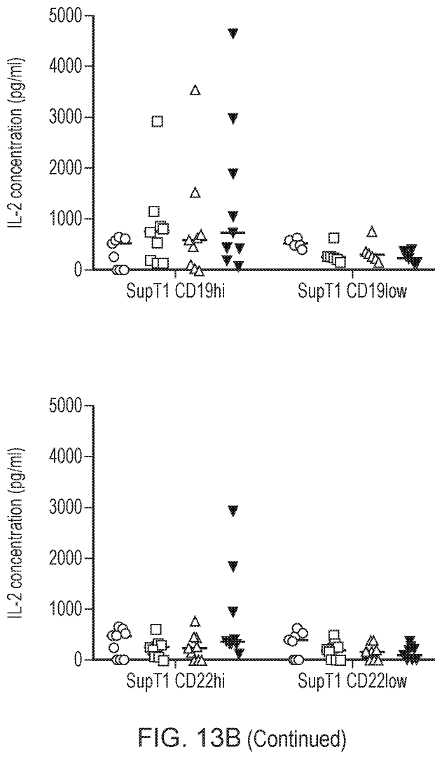

Results are shown in FIG. 13. Construct 5 showed improved killing of low density CD22 target cells. Cytokine production levels were similar.

### Example 4—Module Testing

dnTGFβRII

Cells transduced with Construct 5 were tested for the effect of the dnTGFβRII module when cells are cultured in the presence of TGF-β. Co-cultures with target cells were set up in the presence of rhTGF-β (10 ng/ml) at an E:T ratio of 1:8. Readouts were taken at 7 days. Additionally, effector cells were CTV labelled for proliferation tracking.

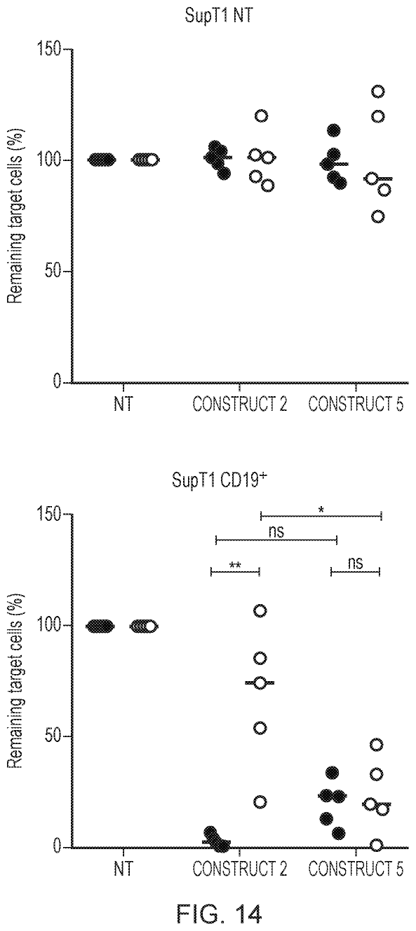

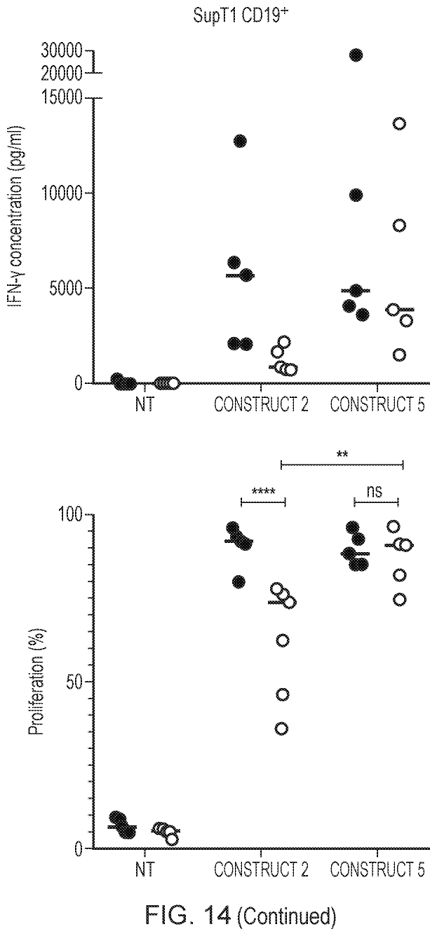

Results are shown in FIG. 14. These data demonstrate that the presence of the dnTGFβRII module improves target cell killing in the presence of TGF-β. Furthermore, the dnTGFβRII module prevents inhibition of proliferation in the presence of TGFβ.

dSHP2

Cells transduced with Construct 5 were tested for the effect of the presence of the dSHP2 module. PBMCs were co-transduced with both Construct 5 and PD1 and then cultured in the presence of cells expressing PDL1. If dSHP2 if effective then its presence will prevent signalling via PD1/PDL1.

Co-cultures with CD19+ target cells, both with and without PDL1 were set up at an E:T ratio of 1:1. Readouts were taken at 6 days.

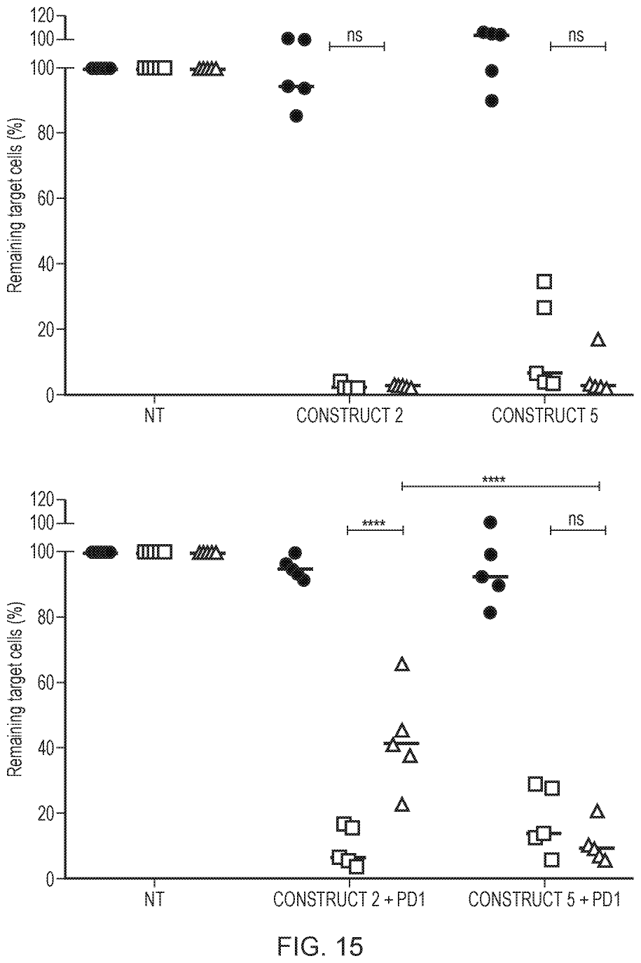

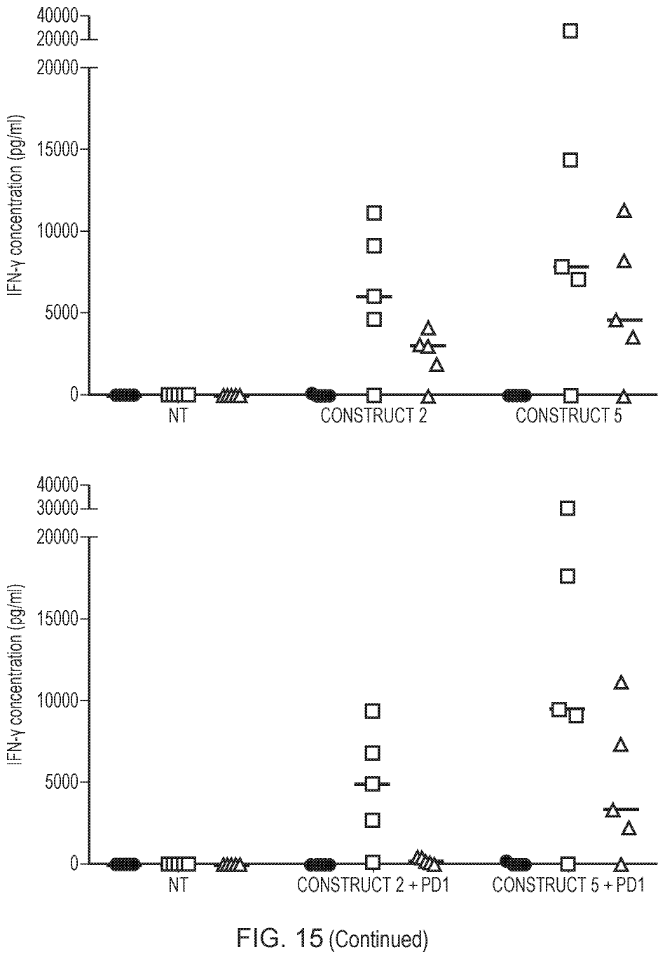

Results are shown in FIG. 15.

These data demonstrate that the presence of dSHP2 overcomes PD1/PDL1 interaction.

### Example 5—Re-stimulation Assay

The performance of Constructs 1, 2, and 5 was investigated using a re-stimulation assay.

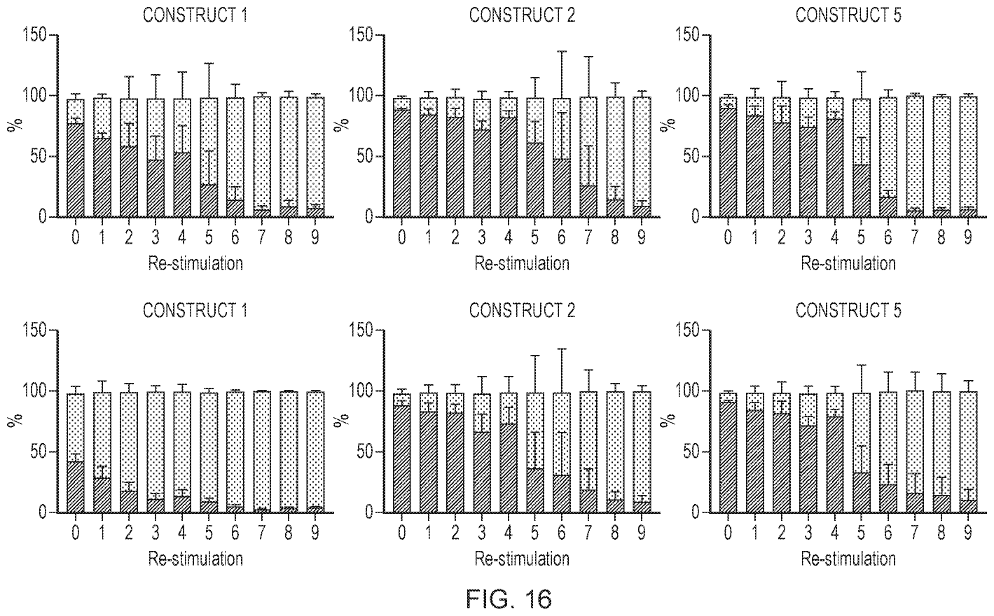

Briefly, CAR-T cells expressing either Construct 1, Construct 2 or Construct 5 were challenged with either CD19+ SupT1 cells or CD22+ SupT1 cells. Plates were re-stimulated with fresh target cells and fresh media every 3 to 4 days, for a total of 9 rounds. The results are shown in FIG. 16.

Both the Construct 2 and Construct 5 expressing CAR-T cells were a greater proportion of the cell population upon re-stimulation, indicating increased target killing. In particular, Construct 2 and Construct 5 expressing cells were a greater proportion of the cell population when CD22 positive target cells were used. These variants therefore show enhanced killing of CD22 positive cells compared to Construct 1.

### Example 6—In Vitro Testing

The ability of T cells transduced with Constructs 3 and 5 to clear tumour cells in a Nalm6 tumour model in NGS mice was investigated. In all cases, mice were injected with 1×10⁶ target cells, NT cells, or PBS on day −6.

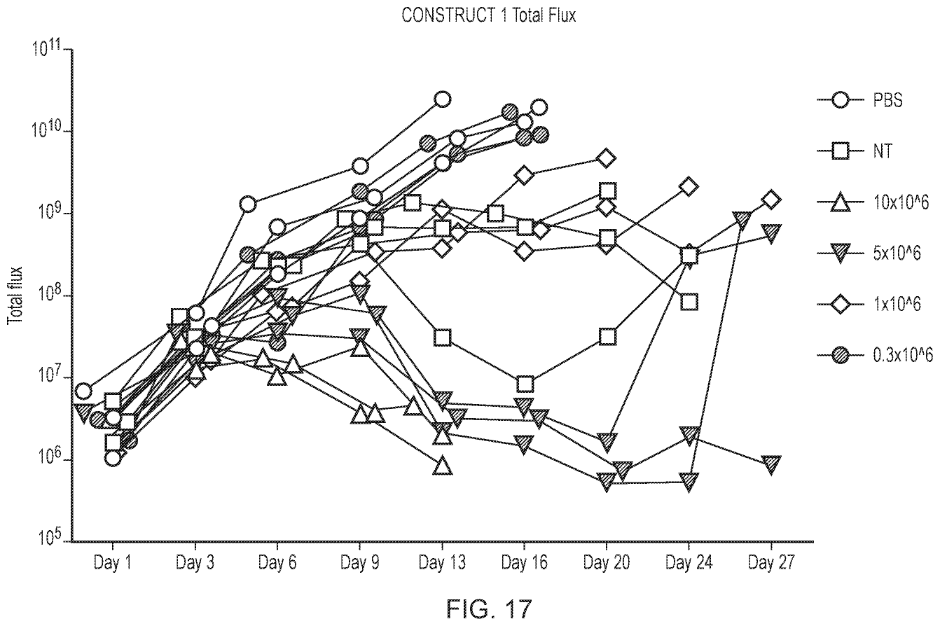

As an initial step, a sub-optimal dose of cells expressing Construct 1 was identified to act as a starting point for Construct 5 dosing. Doses of 0.3×10⁶, 1×10⁶, 5×10⁶, and 10×10⁶ cells were investigated. Results are shown in FIG. 17. The 0.3×10⁶ dose showed similar flux to the PBS control cohort, indicating an inefficient dosage. Clearance was achieved at the 10×10⁶ dose, but mice were sacrificed at day 13 due to suspected Graft versus Host disease (GvH). The 5×10⁶ cohort eliminated target cells, whereas the 1×10⁶ cohort could not control total flux. Following this investigation, a dose of 2.5×10⁶ cells was chosen for testing Constructs 3 and 5.

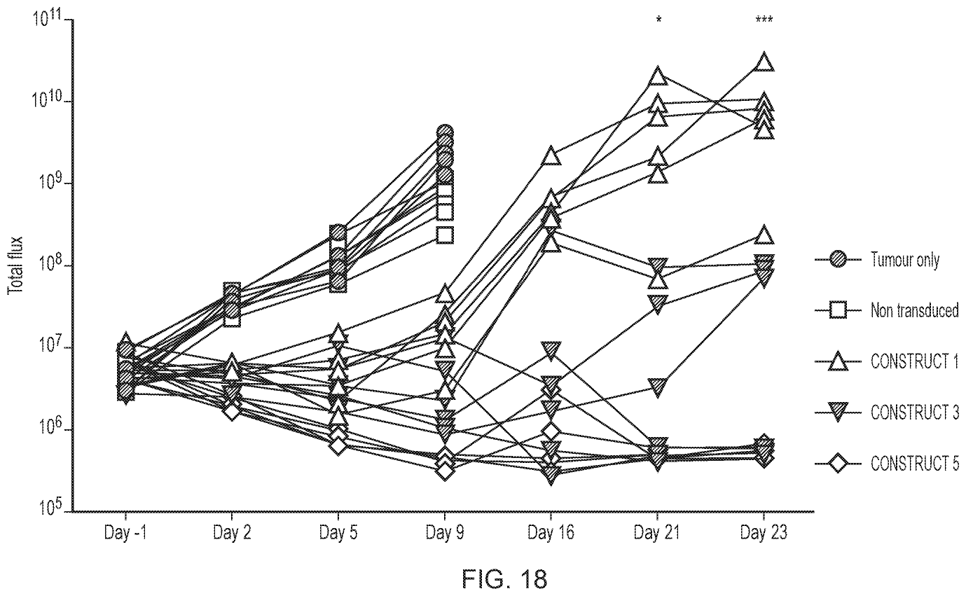

Accordingly, mice were injected with 2.5×10⁶ cells expressing either Construct 1, 3, or 5. Total flux is shown in FIG. 18. Cells expressing Construct 1 are unable to control target cell growth at the 2.5×10⁶ cell dose. Constructs 3 and 5 both show improved function in vivo. In particular, Construct 5 was able to control tumour cell growth to day 23 in all mice. The difference in flux is statistically significant compared to Construct 1.

In addition, Construct 1, 3, or 5 were tested in Nalm6 mice in which CD19 expression has been knocked out (CD19KO). The same conditions as for wild type (WT) Nalm6 mice described above were used, using a 2.5×10⁶ cell dose.

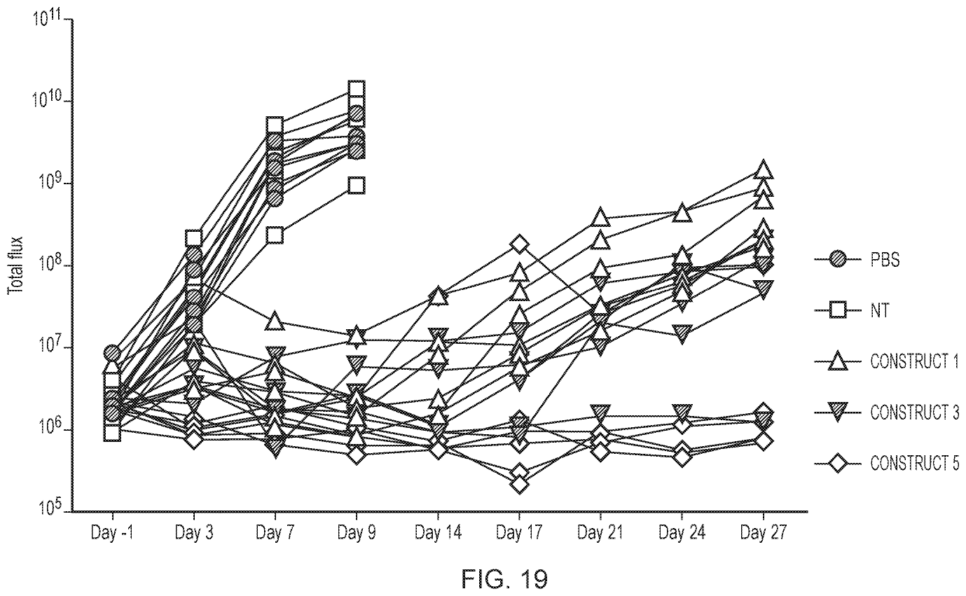

Total flux is shown in FIG. 19. Cells expressing Construct 1 are unable to control target cell growth at the 2.5×10⁶ cell dose. Constructs 3 and 5 both show improved function. In particular, Construct 5 was able to control tumour cell growth to day 27 in all but one mice. These data confirm that cells expressing Construct 5 are able to control tumour burden even in the absence of CD19.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology, cell biology or related fields are intended to be within the scope of the following claims.

1. A cell which co-expresses:
(i) a first chimeric antigen receptor (CAR) at the cell surface, comprising an antigen-binding domain which binds to CD19;
(ii) a second CAR at the cell surface, comprising an antigen-binding domain which binds to CD22;
(iii) dominant negative SHP2 (dSHP2); and
(iv) dominant negative TGFβ receptor II (dnTGFβRII).

2. A cell according to claim 1, wherein each CAR comprises an intracellular signalling domain, wherein the intracellular signalling domain of the first CAR comprises a TNF receptor family endodomain; and the intracellular signalling domain of the second CAR comprises a co-stimulatory endodomain.

3. A cell according to claim 2, wherein the co-stimulatory domain is CD28 co-stimulatory endodomain.

4. A cell according to claim 2, wherein the TNF receptor family endodomain is OX-40 or 4-1BB endodomain.

5. A cell according to claim 2, wherein the intracellular signalling domain of the first and the second CAR also comprises an ITAM-containing domain.

6. A cell according to claim 1, wherein each CAR comprises:
(i) an antigen-binding domain;
(ii) a spacer; and
(iii) a trans-membrane domain;
wherein the spacer of the first CAR is different to the spacer of the second CAR.

7. A cell according to claim 6, wherein the spacer of the second CAR comprises cartilage oligomeric matrix protein (COMP) coiled coil domain.

8. A cell according to claim 1, wherein the first CAR comprises a CD19-binding domain which comprises
a) a heavy chain variable region (VH) having complementarity determining regions (CDRs) with the following sequences:
CDR1-
(SEQ ID No. 1)
SYWMN;
CDR2-
(SEQ ID NO. 2)
QIWPGDGDTNYNGKFK
CDR3-
(SEQ ID No. 3)
RETTTVGRYYYAMDY;
and
b) a light chain variable region (VL) having CDRs with the following sequences:
CDR1-
(SEQ ID No. 4)
KASQSVDYDGDSYLN;
CDR2-
(SEQ ID No. 5)
DASNLVS
CDR3-
(SEQ ID NO. 6)
QQSTEDPWT

9. A cell according to claim 8, wherein the CD19 binding domain comprises a VH domain having the sequence shown as SEQ ID No. 7, or SEQ ID NO 8; or a VL domain having the sequence shown as SEQ ID No 9, SEQ ID No. 10 or SEQ ID No. 11 a variant thereof having at least 90% sequence identity which retains the capacity to bind CD19.

10. A cell according to claim 9, wherein the CD19 binding domain comprises the sequence shown as SEQ ID No 12, SEQ ID No. 13 or SEQ ID No. 14 or a variant thereof having at least 90% sequence identity which retains the capacity to bind CD19.

11. A cell according to claim 1, wherein the second CAR comprises a CD22-binding domain which comprises
a) a heavy chain variable region (VH) having complementarity determining regions (CDRs) with the following sequences:
CDR1-
(SEQ ID No. 15)
NYWIN;
CDR2-
(SEQ ID NO. 16)
NIYPSDSFTNYNQKFKD
CDR3-
(SEQ ID No. 17)
DTQERSWYFDV;
and
b) a light chain variable region (VL) having CDRs with the following sequences:
CDR1-
(SEQ ID No. 18)
RSSQSLVHSNGNTYLH;
CDR2-
(SEQ ID No. 19)
KVSNRFS
CDR3-
(SEQ ID No. 20)
SQSTHVPWT.

12. A cell according to claim 11, wherein the CD22 binding domain comprises a VH domain having the sequence shown as SEQ ID No. 21, or SEQ ID NO 22; or a VL domain having the sequence shown as SEQ ID No 23, or SEQ ID No. 24 or a variant thereof having at least 90% sequence identity which retains the capacity to bind CD22.

13. A cell according to claim 11, wherein the CD22 binding domain comprises the sequence shown as SEQ ID No 25 or SEQ ID No. 26 or a variant thereof having at least 90% sequence identity which retains the capacity to bind CD22.

14. A cell according to claim 1, wherein the first CAR has the structure:
AgB1-spacer1-TM1-TNF-ITAM
in which:
AgB1 is the antigen-binding domain of the first CAR;
spacer1 is the spacer of the first CAR;
TM1 is the transmembrane domain of the first CAR;
TNF is a TNF receptor endodomain; and
ITAM is an ITAM-containing endodomain;
and the second CAR has the structure:
AgB2-spacer2-TM2-costim-ITAM
in which:
AgB2 is the antigen-binding domain of the second CAR;
spacer2 is the spacer of the second CAR;
TM2 is the transmembrane domain of the second CAR;
costim is a co-stimulatory domain; and
ITAM is an ITAM-containing endodomain.

15. A nucleic acid sequence encoding both the first and second chimeric antigen receptors (CARs) as defined in claim 1, dSHP2, and dnTGFβRII.

16. A nucleic acid sequence according to claim 15, which has the following structure:
module1-coexpr-AgB1-spacer1-TM1-coexpr-AgB2-spacer2-TM2-coexpr-module2
in which
AgB1 is a nucleic acid sequence encoding the antigen-binding domain of the first CAR;
spacer1 is a nucleic acid sequence encoding the spacer of the first CAR;
TM1 is a nucleic acid sequence encoding the transmembrane domain of the first CAR;
coexpr is a nucleic acid sequence enabling co-expression
AgB2 is a nucleic acid sequence encoding the antigen-binding domain of the second CAR;
spacer2 is a nucleic acid sequence encoding the spacer of the second CAR;
TM2 is a nucleic acid sequence encoding the transmembrane domain of the second CAR;
module1 and module2 are nucleic acid sequences encoding either dominant negative SHP2 (dSHP2) or dominant negative TGFβRII (dnTGFβRII), wherein when module1 encodes dSHP2 module2 encodes dnTGFβRII and when module2 encodes dnTGFβRII module1 encodes dSHP2;
which nucleic acid sequence, when expressed in a T cell, encodes a polypeptide which is cleaved at the cleavage site such that the first and second CARs are co-expressed at the T cell surface.

17. A nucleic acid sequence according to claim 16, wherein coexpr encodes a sequence comprising a self-cleaving peptide.

18. A nucleic acid sequence according to claim 16, wherein alternative codons are used in regions of sequence encoding the same or similar amino acid sequences, in order to avoid homologous recombination.

19. (canceled)

20. A retroviral vector or a lentiviral vector or a transposon comprising a nucleic aid sequence according to claim 15.

21. A method for making a cell according to claim 1, which comprises the step of introducing: a nucleic acid sequence according to claim 15; or a vector according to claim 20, into a cell.

22. A method according to claim 21, wherein the cell is from a sample isolated from a subject.

23. A pharmaceutical composition comprising a plurality of cells according to claim 1.

24. A method for treating and/or preventing a disease, which comprises the step of administering a pharmaceutical composition according to claim 23 to a subject.

25. A method according to claim 24, which comprises the following steps:
(i) isolation of a cell-containing sample from a subject;
(ii) transduction or transfection of the cells with: a nucleic acid sequence according to any of claims 15 to 18; or a vector according to claim 19 or 20; and
(iii) administering the cells from (ii) to the subject.

26. A method according to claim 24, wherein the disease is a cancer.

27. A method according to claim 26, wherein the cancer is a B cell malignancy.

28-29. (canceled)

30. A kit which comprises
(i) a first nucleic acid sequence encoding the first chimeric antigen receptor (CAR), which nucleic acid sequence has the following structure:
AgB1-spacer1-TM1
in which
AgB1 is a nucleic acid sequence encoding the antigen-binding domain of the first CAR which binds to CD19;
spacer1 is a nucleic acid sequence encoding the spacer of the first CAR;
TM1 is a nucleic acid sequence encoding the transmembrane domain of the first CAR;
(ii) a second nucleic acid sequence encoding the second chimeric antigen receptor, which nucleic acid sequence has the following structure:
AgB2-spacer2-TM2
in which
AgB2 is a nucleic acid sequence encoding the antigen-binding domain of the second CAR which binds to CD22;
spacer2 is a nucleic acid sequence encoding the spacer of the second CAR; and
TM2 is a nucleic acid sequence encoding the transmembrane domain of the second CAR; and
(iii) a third nucleic acid sequence encoding dSHP2 and dnTGFβRII as described herein.

31. A kit comprising: a first vector which comprises the first nucleic acid sequence as defined in claim 30; a second vector which comprises the second nucleic acid sequence as defined in claim 30; and a third vector which comprises the third nucleic acid sequence as defined in claim 30.


SEQUENCE LISTING (106 sequences)

SEQ ID NO: 1 - protein, 5 aa, Artificial
  Synthetic construct
SYWMN

SEQ ID NO: 2 - protein, 16 aa, Artificial
  Synthetic construct
QIWPGDGDTNYNGKFK

SEQ ID NO: 3 - protein, 15 aa, Artificial
  Synthetic construct
RETTTVGRYYYAMDY

SEQ ID NO: 4 - protein, 15 aa, Artificial
  Synthetic construct
KASQSVDYDGDSYLN

SEQ ID NO: 5 - protein, 7 aa, Artificial
  Synthetic construct
DASNLVS

SEQ ID NO: 6 - protein, 9 aa, Artificial
  Synthetic construct
QQSTEDPWT

SEQ ID NO: 7 - protein, 124 aa, Artificial
  Synthetic construct
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 8 - protein, 124 aa, Artificial
  Synthetic construct
QVQLVQSGAEVKKPGASVKLSCKASGYAFSSYWMNWVRQAPGQSLEWIGQIWPGDGDTNYNGKFKGRATLTADESARTAYMELSSLRSGDTAVYFCARRETTTVGRYYYAMDYWGKGTLVTVSS

SEQ ID NO: 9 - protein, 111 aa, Artificial
  Synthetic construct
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 10 - protein, 112 aa, Artificial
  Synthetic construct
DIQLTQSPDSLAVSLGERATINCKASQSVDYDGDSYLNWYQQKPGQPPKLLIYDASNLVSGVPDRFSGSGSGTDFTLTISSLQAADVAVYHCQQSTEDPWTFGQGTKVEIKR

SEQ ID NO: 11 - protein, 112 aa, Artificial
  Synthetic construct
DIQLTQSPDSLAVSLGERATINCKASQSVDYDGDSYLNWYQQKPGQPPKVLIYDASNLVSGVPDRFSGSGSGTDFTLTISSLQAADVAVYYCQQSTEDPWTFGQGTKVEIKR

SEQ ID NO: 12 - protein, 235 aa, Artificial
  Synthetic construct
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 13 - protein, 236 aa, Artificial
  Synthetic construct
QVQLVQSGAEVKKPGASVKLSCKASGYAFSSYWMNWVRQAPGQSLEWIGQIWPGDGDTNYNGKFKGRATLTADESARTAYMELSSLRSGDTAVYFCARRETTTVGRYYYAMDYWGKGTLVTVSSDIQLTQSPDSLAVSLGERATINCKASQSVDYDGDSYLNWYQQKPGQPPKLLIYDASNLVSGVPDRFSGSGSGTDFTLTISSLQAADVAVYHCQQSTEDPWTFGQGTKVEIKR

SEQ ID NO: 14 - protein, 236 aa, Artificial
  Synthetic construct
QVQLVQSGAEVKKPGASVKLSCKASGYAFSSYWMNWVRQAPGQSLEWIGQIWPGDGDTNYNGKFKGRATLTADESARTAYMELSSLRSGDTAVYFCARRETTTVGRYYYAMDYWGKGTLVTVSSDIQLTQSPDSLAVSLGERATINCKASQSVDYDGDSYLNWYQQKPGQPPKVLIYDASNLVSGVPDRFSGSGSGTDFTLTISSLQAADVAVYYCQQSTEDPWTFGQGTKVEIKR

SEQ ID NO: 15 - protein, 5 aa, Artificial
  Synthetic construct
NYWIN

SEQ ID NO: 16 - protein, 17 aa, Artificial
  Synthetic construct
NIYPSDSFTNYNQKFKD

SEQ ID NO: 17 - protein, 11 aa, Artificial
  Synthetic construct
DTQERSWYFDV

SEQ ID NO: 18 - protein, 16 aa, Artificial
  Synthetic construct
RSSQSLVHSNGNTYLH

SEQ ID NO: 19 - protein, 7 aa, Artificial
  Synthetic construct
KVSNRFS

SEQ ID NO: 20 - protein, 9 aa, Artificial
  Synthetic construct
SQSTHVPWT

SEQ ID NO: 21 - protein, 120 aa, Artificial
  Synthetic construct
QVQLQQPGAELVRPGASVKLSCKASGYTFTNYWINWVKQRPGQGLEWIGNIYPSDSFTNYNQKFKDKATLTVDKSSSTAYMQLSSPTSEDSAVYYCTRDTQERSWYFDVWGAGTTVTVSS

SEQ ID NO: 22 - protein, 120 aa, Artificial
  Synthetic construct
EVQLVESGAEVKKPGSSVKVSCKASGYTFTNYWINWVRQAPGQGLEWIGNIYPSDSFTNYNQKFKDRATLTVDKSTSTAYLELRNLRSDDTAVYYCTRDTQERSWYFDVWGQGTLVTVSS

SEQ ID NO: 23 - protein, 112 aa, Artificial
  Synthetic construct
DVVMTQTPLSLPVSLGDQASISCRSSQSLVHSNGNTYLHWYLQKPGQSPKLLIYKVSNRFSGVPDRFSGSGSGTDFTLKISRVEAEDLGLYFCSQSTHVPWTFGGGTKLEIK

SEQ ID NO: 24 - protein, 112 aa, Artificial
  Synthetic construct
DIVMTQSPATLSVSPGERATLSCRSSQSLVHSNGNTYLHWYQQKPGQAPRLLIYKVSNRFSGVPARFSGSGSGVEFTLTISSLQSEDFAVYYCSQSTHVPWTFGQGTRLEIK

SEQ ID NO: 25 - protein, 232 aa, Artificial
  Synthetic construct
QVQLQQPGAELVRPGASVKLSCKASGYTFTNYWINWVKQRPGQGLEWIGNIYPSDSFTNYNQKFKDKATLTVDKSSSTAYMQLSSPTSEDSAVYYCTRDTQERSWYFDVWGAGTTVTVSSDVVMTQTPLSLPVSLGDQASISCRSSQSLVHSNGNTYLHWYLQKPGQSPKLLIYKVSNRFSGVPDRFSGSGSGTDFTLKISRVEAEDLGLYFCSQSTHVPWTFGGGTKLEIK

SEQ ID NO: 26 - protein, 232 aa, Artificial
  Synthetic construct
EVQLVESGAEVKKPGSSVKVSCKASGYTFTNYWINWVRQAPGQGLEWIGNIYPSDSFTNYNQKFKDRATLTVDKSTSTAYLELRNLRSDDTAVYYCTRDTQERSWYFDVWGQGTLVTVSSDIVMTQSPATLSVSPGERATLSCRSSQSLVHSNGNTYLHWYQQKPGQAPRLLIYKVSNRFSGVPARFSGSGSGVEFTLTISSLQSEDFAVYYCSQSTHVPWTFGQGTRLEIK

SEQ ID NO: 27 - protein, 21 aa, Artificial
  Synthetic construct
MGTSLLCWMALCLLGADHADG

SEQ ID NO: 28 - protein, 21 aa, Artificial
  Synthetic construct
MSLPVTALLLPLALLLHAARP

SEQ ID NO: 29 - protein, 20 aa, Artificial
  Synthetic construct
MAVPTQVLGLLLLWLTDARC

SEQ ID NO: 30 - protein, 7 aa, Artificial
  Synthetic construct
GYAFSSS

SEQ ID NO: 31 - protein, 6 aa, Artificial
  Synthetic construct
YPGDED

SEQ ID NO: 32 - protein, 10 aa, Artificial
  Synthetic construct
SLLYGDYLDY

SEQ ID NO: 33 - protein, 10 aa, Artificial
  Synthetic construct
SASSSVSYMH

SEQ ID NO: 34 - protein, 7 aa, Artificial
  Synthetic construct
DTSKLAS

SEQ ID NO: 35 - protein, 9 aa, Artificial
  Synthetic construct
QQWNINPLT

SEQ ID NO: 36 - protein, 119 aa, Artificial
  Synthetic construct
QVQLQQSGPELVKPGASVKISCKASGYAFSSSWMNWVKQRPGKGLEWIGRIYPGDEDTNYSGKFKDKATLTADKSSTTAYMQLSSLTSEDSAVYFCARSLLYGDYLDYWGQGTTLTVSS

SEQ ID NO: 37 - protein, 107 aa, Artificial
  Synthetic construct
QIVLTQSPAIMSASPGEKVTMTCSASSSVSYMHWYQQKSGTSPKRWIYDTSKLASGVPDRFSGSGSGTSYFLTINNMEAEDAATYYCQQWNINPLTFGAGTKLELKR

SEQ ID NO: 38 - protein, 241 aa, Artificial
  Synthetic construct
QVQLQQSGPELVKPGASVKISCKASGYAFSSSWMNWVKQRPGKGLEWIGRIYPGDEDTNYSGKFKDKATLTADKSSTTAYMQLSSLTSEDSAVYFCARSLLYGDYLDYWGQGTTLTVSSGGGGSGGGGSGGGGSQIVLTQSPAIMSASPGEKVTMTCSASSSVSYMHWYQQKSGTSPKRWIYDTSKLASGVPDRFSGSGSGTSYFLTINNMEAEDAATYYCQQWNINPLTFGAGTKLELKR

SEQ ID NO: 39 - protein, 119 aa, Artificial
  Synthetic construct
EVQLVESGGGLVQPKGSLKLSCAASGFTFNTYAMHWVRQAPGKGLEWVARIRSKSSNYATYYADSVKDRFTISRDDSQSMLYLQMNNLKTEDTAMYYCVVDYLYAMDYWGQGTSVTVSS

SEQ ID NO: 40 - protein, 107 aa, Artificial
  Synthetic construct
DIVMTQSQKFMSTSVGDRVSITCKASQNVRTAVAWYQQKPGQSPKALIYLASNRHTGVPDRFTGSGSGTDFTLTISNVQSEDLADYFCLQHWNYPFTFGSGTKLEIK

SEQ ID NO: 41 - protein, 120 aa, Artificial
  Synthetic construct
QVQLQQSGAELVRPGASVTLSCKASGYTFTDYEMHWVKQTPVHGLEWIGAIDPETGATAYNQKFKGKAILTADKSSSTAYMDLRSLTSEDSAVYYCTRYDYGSSPWFAYWGQGTLVTVSA

SEQ ID NO: 42 - protein, 109 aa, Artificial
  Synthetic construct
QAVVTQESALTTSPGETVTLTCRSSAGAVTTSNYANWVQEKPDHLFTGLIGGTNNRAPGVPARFSGSLIGDKAALTITGAQTEDEAIYFCALWNSNHWVFGGGTKLTVL

SEQ ID NO: 43 - protein, 119 aa, Artificial
  Synthetic construct
QVQLQQPGAELVMPGASVKLSCKASGYTFTSYWMHWVKQRPGQGLEWIGEIDPSDSYTNYNQKFKGKATLTVDKSSSTAYMQLSSLTSEDSAVYYCARGYYGSSSFDYWGQGTTLTVSS

SEQ ID NO: 44 - protein, 111 aa, Artificial
  Synthetic construct
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYTFGGGTKLEIK

SEQ ID NO: 45 - protein, 120 aa, Artificial
  Synthetic construct
QVQLKESGPGLVAPSQSLSITCTVSGFSLTSYGVHWVRQPPGKGLEWLVVIWSDGSTTYNSALKSRLSISKDNSKSQVFLKMNSLQTDDTAMYYCARHADDYGFAWFAYWGQGTLVTVSA

SEQ ID NO: 46 - protein, 107 aa, Artificial
  Synthetic construct
DIQMTQSPASLSASVGETVTITCRASENIYSYLAWYQQKQGKSPQLLVYNAKTLAEGVPSRFSGSGSGTQFSLKINSLQPEDFGSYYCQHHYGTPPTFGGGTKLEIK

SEQ ID NO: 47 - protein, 121 aa, Artificial
  Synthetic construct
EFQLQQSGPELVKPGASVKISCKASGYSFTDYNMNWVKQSNGKSLEWIGVINPNYGTTSYNQKFKGKATLTVDQSSSTAYMQLNSLTSEDSAVYYCARSSTTVVDWYFDVWGTGTTVTVSS

SEQ ID NO: 48 - protein, 107 aa, Artificial
  Synthetic construct
DIQMTQSPSSLSASLGERVSLTCRASQEISGYLSWLQQKPDGTIKRLIYAASTLDSGVPKRFSGSRSGSDYSLTISSLESEDFADYYCLQYASYPFTFGSGTKLEIK

SEQ ID NO: 49 - protein, 118 aa, Artificial
  Synthetic construct
QVQVQQPGAELVRPGTSVKLSCKASGYTFTRYWMYWVKQRPGQGLEWIGVIDPSDNFTYYNQKFKGKATLTVDTSSSTAYMQLSSLTSEDSAVYYCARGYGSSYVGYWGQGTTLTVSS

SEQ ID NO: 50 - protein, 113 aa, Artificial
  Synthetic construct
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPPWTFGGGTKLEIK

SEQ ID NO: 51 - protein, 124 aa, Artificial
  Synthetic construct
QVTLKESGPGILQSSQTLSLTCSFSGFSLSTSDMGVSWIRQPSGKGLEWLAHIYWDDDKRYNPSLKSRLTISKDASRNQVFLKIATVDTADTATYYCARSPWIYYGHYWCFDVWGTGTTVTVSS

SEQ ID NO: 52 - protein, 107 aa, Artificial
  Synthetic construct
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPFTFGSGTKLEIK

SEQ ID NO: 53 - protein, 123 aa, Artificial
  Synthetic construct
QVQLQQSGAELVKPGASVKLSCKASGYTFTEYTIHWVKQRSGQGLEWIGWFYPGSGSIKYNEKFKDKATLTADKSSSTVYMELSRLTSEDSAVYFCARHGDGYYLPPYYFDYWGQGTTLTVSS

SEQ ID NO: 54 - protein, 106 aa, Artificial
  Synthetic construct
QIVLTQSPAIMSASPGEKVTMTCSASSSVSYMYWYQQKPGSSPRLLIYDTSNLASGVPVRFSGSGSGTSYSLTISRMEAEDAATYYCQQWSSYPLTFGAGTKLELK

SEQ ID NO: 55 - protein, 121 aa, Artificial
  Synthetic construct
QVQLQQSGAELARPGASVKLSCKASGYIFTSYGISWVKQRTGQGLEWIGEIYPRSGNTYYNEKFKGKATLTADKSSSTAYMELRSLTSEDSAVYFCARPIYYGSREGFDYWGQGTTLTVSS

SEQ ID NO: 56 - protein, 107 aa, Artificial
  Synthetic construct
DIVLTQSPATLSVTPGDSVSLSCRASQSISTNLHWYQQKSHASPRLLIKYASQSVSGIPSRFSGSGSGTDFTLSINSVETEDFGIFFCQQSYSWPYTFGGGTKLEIK

SEQ ID NO: 57 - protein, 120 aa, Artificial
  Synthetic construct
QVQLQQPGAELVMPGASVKLSCKASGYTFTSYWMHWVKQRPGQGLEWIGEIDPSDSYTNYNQKFKGKSTLTVDKSSSTAYIQLSSLTSEDSAVYYCARWASYRGYAMDYWGQGTSVTVSS

SEQ ID NO: 58 - protein, 112 aa, Artificial
  Synthetic construct
DVLMTQTPLSLPVSLGDQASISCRSSQSIVHSNGNTYLEWYLQKPGQSPKLLIYKVSNRFSGVPDRFSGSESGTDFTLKISRVEAEDLGVYYCFQGSHVPWTFGGGTKLEIK

SEQ ID NO: 59 - protein, 119 aa, Artificial
  Synthetic construct
EFQLQQSGPELVKPGASVKISCKASGYSFTDYNMNWVKQSNGKSLEWIGVINPNYGTTSYNQRFKGKATLTVDQSSSTAYMQLNSLTSEDSAVYYCARSGLRYWYFDVWGTGTTVTVSS

SEQ ID NO: 60 - protein, 107 aa, Artificial; identical to SEQ ID NO: 48
  Synthetic construct

SEQ ID NO: 61 - protein, 121 aa, Artificial
  Synthetic construct
EVQLVQSGAEVKKPGASVKVSCKASGYRFTNYWIHWVRQAPGQGLEWIGGINPGNNYATYRRKFQGRVTMTADTSTSTVYMELSSLRSEDTAVYYCTREGYGNYGAWFAYWGQGTLVTVSS

SEQ ID NO: 62 - protein, 113 aa, Artificial
  Synthetic construct
DVQVTQSPSSLSASVGDRVTITCRSSQSLANSYGNTFLSWYLHKPGKAPQLLIYGISNRFSGVPDRFSGSGSGTDFTLTISSLQPEDFATYYCLQGTHQPYTFGQGTKVEIKR

SEQ ID NO: 63 - protein, 127 aa, Artificial
  Synthetic construct
EVQLVESGGGLVQPGRSLKLSCAASGFTFSNFAMAWVRQPPTKGLEWVASISTGGGNTYYRDSVKGRFTISRDDAKNTQYLQMDSLRSEDTATYYCARQRNYYDGSYDYEGYTMDAWGQGTSVTVSS

SEQ ID NO: 64 - protein, 107 aa, Artificial
  Synthetic construct
DIQMTQSPSSLSASLGDRVTITCRSSQDIGNYLTWFQQKVGRSPRRMIYGAIKLEDGVPSRFSGSRSGSDYSLTISSLESEDVADYQCLQSIQYPFTFGSGTKLEIK

SEQ ID NO: 65 - protein, 234 aa, Artificial
  Synthetic construct
AEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKD

SEQ ID NO: 66 - protein, 46 aa, Artificial
  Synthetic construct
TTTPAPRPPTPAPTIASQPLSLRPEACRPAAGGAVHTRGLDFACDI

SEQ ID NO: 67 - protein, 20 aa, Artificial
  Synthetic construct
AEPKSPDKTHTCPPCPKDPK

SEQ ID NO: 68 - protein, 185 aa, Artificial
  Synthetic construct
KEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLD

SEQ ID NO: 69 - protein, 259 aa, Artificial
  Synthetic construct
SLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKT

SEQ ID NO: 70 - protein, 57 aa, Artificial
  Synthetic construct
MHAALSTEVVHLRQRTEELLRCNEQQAAELETCKEQLFQSNMERKELHNTVMDLRGN

SEQ ID NO: 71 - protein, 50 aa, Artificial [sequence omitted]

SEQ ID NO: 72 - protein, 61 aa, Artificial
  Synthetic construct
RYVVALVKALEEIDESINMLNEKLEDIRAVKESEITEKFEKKIRELRELRRDVEREIEEVM

SEQ ID NO: 73 - protein, 31 aa, Artificial
  Synthetic construct
AIEVKLANMEAEINTLKSKLELTNKLHAFSM

SEQ ID NO: 74 - protein, 29 aa, Artificial
  Synthetic construct
EWEALEKKLAALESKLQALEKKLEALEHG

SEQ ID NO: 75 - protein, 24 aa, Artificial
  Synthetic construct
INPVNNRIQDLTERSDVLRGYLDY

SEQ ID NO: 76 - protein, 51 aa, Artificial
  Synthetic construct
VVDTLDQMKQGLEDVSGLLELAVEADDEETFNEAVAELDALEEKLAQLEFR

SEQ ID NO: 77 - protein, 51 aa, Artificial
  Synthetic construct
IETRHSEIIKLENSIRELHDMFMDMAMLVESQGEMIDRIEYNVEHAVDYVE

SEQ ID NO: 78 - protein, 67 aa, Artificial
  Synthetic construct
ALSEIETRHSEIIKLENSIRELHDMFMDMAMLVESQGEMIDRIEYNVEHAVDYVERAVSDTKKAVKY

SEQ ID NO: 79 - protein, 69 aa, Artificial
  Synthetic construct
ELEEMQRRADQLADESLESTRRMLQLVEESKDAGIRTLVMLDEQGEQLERIEEGMDQINKDMKEAEKNL

SEQ ID NO: 80 - protein, 51 aa, Artificial; identical to SEQ ID NO: 77
  Synthetic construct

SEQ ID NO: 81 - protein, 20 aa, Artificial
  Synthetic construct
SPRALADSLMQLARQVSRLE

SEQ ID NO: 82 - protein, 136 aa, Artificial
  Synthetic construct
SGQRWELALGRFWDYLRWVQTLSEQVQEELLSSQVTQELRALMDETMKELKAYKSELEEQLTARLSKELQAAQARLGADMEDVCGRLVQYRGEVQAMLGQSTEELRVRLASHLRKLRKRLLRDADDLQKRLAVYQA

SEQ ID NO: 83 - protein, 45 aa, Artificial
  Synthetic construct
DLGPQMLRELQETNAALQDVRELLRQQVREITFLKNTVMECDACG

SEQ ID NO: 84 - protein, 20 aa, Artificial
  Synthetic construct
QQVREITFLKNTVMECDACG

SEQ ID NO: 85 - protein, 24 aa, Artificial
  Synthetic construct
IIAIAVVGALLLVALIFGTASYLI

SEQ ID NO: 86 - protein, 140 aa, Artificial
  Synthetic construct
FWVLVVVGGVLACYSLLVTVAFIIFWVRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 87 - protein, 180 aa, Artificial
  Synthetic construct
FWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 88 - protein, 216 aa, Artificial
  Synthetic construct
FWVLVVVGGVLACYSLLVTVAFIIFWVRSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 89 - protein, 40 aa, Artificial
  Synthetic construct
SKRSRLLHSDYMNMTPRRPGPTRKHYQPYAPPRDFAAYRS

SEQ ID NO: 90 - protein, 36 aa, Artificial
  Synthetic construct
RDQRLPPDAHKPPGGGSFRTPIQEEQADAHSTLAKI

SEQ ID NO: 91 - protein, 42 aa, Artificial
  Synthetic construct
KRGRKKLLYIFKQPFMRPVQTTQEEDGCSCRFPEEEEGGCEL

SEQ ID NO: 92 - protein, 112 aa, Artificial
  Synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 93 - protein, 20 aa, Artificial
  Synthetic construct
RAEGRGSLLTCGDVEENPGP

SEQ ID NO: 94 - protein, 214 aa, Artificial
  Synthetic construct
MVRWFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPLNCSDPTSERWYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPYY

SEQ ID NO: 95 - protein, 97 aa, Artificial
  Synthetic construct
WFHRDLSGLDAETLLKGRGVHGSFLARPSRKNQGDFSLSVRVGDQVTHIRIQNSGDFYDLYGGEKFATLTELVEYYTQQQGVLQDRDGTIIHLKYPL

SEQ ID NO: 96 - protein, 104 aa, Artificial
  Synthetic construct
WYHGHMSGGQAETLLQAKGEPWTFLVRESLSQPGDFVLSVLSDQPKAGPGSPLRVTHIKVMCEGGRYTVGGLETFDSLTDLVEHFKKTGIEEASGAFVYLRQPY

SEQ ID NO: 97 - protein, 97 aa, Artificial
  Synthetic construct
WFHPNITGVEAENLLLTRGVDGSFLARPSKSNPGDFTLSVRRNGAVTHIKIQNTGDYYDLYGGEKFATLAELVQYYMEHHGQLKEKNGDVIELKYPL

SEQ ID NO: 98 - protein, 105 aa, Artificial
  Synthetic construct
WFHGHLSGKEAEKLLTEKGKHGSFLVRESQSHPGDFVLSVRTGDDKGESNDGKSKVTHVMIRCQELKYDVGGGERFDSLTDLVEHYKKNPMVETLGTVLQLKQPL

SEQ ID NO: 99 - protein, 211 aa, Artificial
  Synthetic construct
WFHPNITGVEAENLLLTRGVDGSFLARPSKSNPGDFTLSVRRNGAVTHIKIQNTGDYYDLYGGEKFATLAELVQYYMEHHGQLKEKNGDVIELKYPLNCADPTSERWFHGHLSGKEAEKLLTEKGKHGSFLVRESQSHPGDFVLSVRTGDDKGESNDGKSKVTHVMIRCQELKYDVGGGERFDSLTDLVEHYKKNPMVETLGTVLQLKQPL

SEQ ID NO: 100 - protein, 177 aa, Artificial
  Synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSS

SEQ ID NO: 101 - protein, 5 aa, Artificial
  Synthetic construct
NFAMA

SEQ ID NO: 102 - protein, 17 aa, Artificial
  Synthetic construct
SISTGGGNTYYRDSVKG

SEQ ID NO: 103 - protein, 18 aa, Artificial
  Synthetic construct
QRNYYDGSYDYEGYTMDA

SEQ ID NO: 104 - protein, 11 aa, Artificial
  Synthetic construct
RSSQDIGNYLT

SEQ ID NO: 105 - protein, 7 aa, Artificial
  Synthetic construct
GAIKLED

SEQ ID NO: 106 - protein, 7 aa, Artificial
  Synthetic construct
LQSIQYP